(19) United States
(12) Patent Application Publication
(10) Pub. No.: US 2004/0028663 A1
(43) Pub. Date: Feb. 12, 2004

(54) Combined immunotherapy of fusion cells and interleukin-12 for treatment of cancer

(76) Inventor: Tsuneya Ohno, Boston, MA (US)

Correspondence Address: PENNIE AND EDMONDS, 1155 AVENUE OF THE AMERICAS, NEW YORK, NY, 100362711

(21) Appl. No.: 10/328,998

(22) Filed: Dec. 24, 2002

Related U.S. Application Data
(63) Continuation-in-part of application No. 10/012,134, filed on Oct. 22, 2001.
(60) Provisional application No. 60/242,154, filed on Oct. 20, 2000.

(51) Int. Cl. A61K 48/00; A61K 38/20

(52) U.S. Cl. 424/93.21; 424/85.2

(57) ABSTRACT

The present invention relates to methods and compositions for treating and preventing cancer by administering a therapeutically effective dose of fusion cells formed by fusion of autologous dendritic cells and autologous non-dendritic cells, in combination with a cytokine or other molecule which stimulates or induces a cytotoxic T cell response and/or a humoral immune response.

This application is a continuation-in-part of pending U.S. application Ser. No. 10/012,134, filed on Oct. 22, 2001, claiming benefit to U.S. Provisional Application Serial No. 60/242,154, filed Oct. 20, 2000.

### 1. INTRODUCTION

The present invention relates to methods and treatment protocols for the immunotherapy of cancer by administering a therapeutically effective dose of fusion cells formed by fusion of autologous dendritic cells and autologous non-dendritic cells in combination with interleukin-12.

### 2. BACKGROUND OF THE INVENTION

There is great interest in the development of an effective immunotherapeutic composition for treating or preventing cancer. Success at such an immunotherapeutic approach will require the development of a composition that is both capable of eliciting a very strong immune response, and, at the same time, extremely specific for the target tumor or infected cell.

2.1 The Immune Response

Cells of the immune system arise from pluripotent stem cells through two main lines of differentiation, the lymphoid lineage and the myeloid lineage. The lymphoid lineage produces lymphocytes, such as T cells, B cells, and natural killer cells, while the myeloid lineage produces monocytes, macrophages, and neutrophils and other accessory cells, such as dendritic cells, platelets, and mast cells. There are two main types of T cells of the lymphoid lineage, cytotoxic T lymphocytes (“CTLs”) and helper T cells which mature and undergo selection in the thymus, and are distinguished by the presence of one of two surface markers, for example, CD8 (CTLs) or CD4 (helper T cells).

Lymphocytes circulate and search for invading foreign pathogens and antigens that tend to become trapped in secondary lymphoid organs, such as the spleen and the lymph nodes. Antigens are taken up in the periphery by the antigen-presenting cells (APCs) and migrate to secondary organs. Interaction between T cells and APCs triggers several effector pathways, including activation of B cells and antibody production as well as activation of CD8⁺ cytotoxic T lymphocytes (CD8⁺ CTLs) and stimulation of T cell production of cytokines.

CTLs then kill target cells that carry the same class I MHC molecule and the same antigen that originally induced their activation. CD8⁺ CTLs are important in resisting cancer and pathogens, as well as rejecting allografts (Terstappen et al., 1992, Blood 79:666-677).

Antigens are processed by two distinct routes depending upon whether their origin is intracellular or extracellular. Intracellular or endogenous protein antigens are presented to CD8⁺ CTLs by class I major histocompatibility complex (MHC) molecules, expressed in most cell types, including tumor cells. On the other hand, extracellular antigenic determinants are presented on the cell surface of “specialized” or “professional” APCs, such as dendritic cells and macrophages, for example, by class II MHC molecules to CD4⁺ “helper” T cells (see generally, W. E. Paul, ed., Fundamental Immunology. New York: Raven Press, 1984).

Class I and class II MHC molecules are the most polymorphic proteins known. A further degree of heterogeneity of MHC molecules is generated by the combination of class I and class II MHC molecules, known as the MHC haplotype. In humans, HLA-A, HLA-B and HLA-C, three distinct genetic loci located on a single chromosome, encode class I molecules. Because T cell receptors specifically bind complexes comprising antigenic peptides and the polymorphic portion of MHC molecules, T cells respond poorly when an MHC molecule of a different genetic type is encountered. This specificity results in the phenomenon of MHC-restricted T cell recognition and T cell cytotoxicity.

Lymphocytes circulate in the periphery and become “primed” in the lymphoid organs on encountering the appropriate signals (Bretscher and Cohn, 1970, Science 169:1042-1049). The first signal is received through the T cell receptor after it engages antigenic peptides displayed by class I MHC molecules on the surface of APCs. The second signal is provided either by a secreted chemical signal or cytokine, such as interleukin-1 (IL-1), interferon-γ, interleukin-2 (IL-2), interleukin-4 (IL-4), interleukin-7 (IL-7), and interleukin-12 (IL-12), produced by CD4+helper T cells or dendritic cells, or by a plasma-membrane-bound co-stimulatory molecule, such as B7, which is present on the antigen-presenting cell membrane and is recognized by a co-receptor on the cell surface of helper T cells, called CD28, a member of the Ig superfamily. Interferon-γ and IL-12 are associated with the helper T cell subtype known as TH₁, which promote the development of CD8⁺ T cells, and IL-4 is associated with the T helper cell subtype known as TH₂, which promote the development and activation of B cells to produce antibodies.

In addition to antigen-specific interactions during antigen presentation, antigen nonspecific adhesive mechanisms also operate. These stabilize the binding of T lymphocytes to APC. Receptor molecules on APC, such as ICAM-1/CD54, LFA-3/CD58, and B7, bind corresponding co-receptors on T cells.

Thus, helper T cells receiving both signals are activated to proliferate and to secrete a variety of interleukins. CTLs receiving both signals are activated to kill target cells. However, T cells receiving the first signal in the absence of co-stimulation become anergized, leading to tolerance (Lamb et al., 1983, J. Exp. Med. 157:1434-1447; Mueller et al., 1989, Annu. Rev. Immunol. 7:445-480; Schwartz, 1992, Cell 71:1065-1068; Mueller and Jenkins, 1995, Curr. Opin. Immunol. 7:375-381).

2.2 Immunotherapy Against Cancer

The cytotoxic T cell response is the most important host response for the control of growth of antigenic tumor cells (Anichimi et al., 1987, Immunol. Today 8:385-389). Studies with experimental animal tumors as well as spontaneous human tumors have demonstrated that many tumors express antigens that can induce an immune response. Some antigens are unique to the tumor, and some are found on both tumor and normal cells. Several factors influence the immunogenicity of the tumor, including, for example, the specific type of carcinogen involved, and immunocompetence of the host and the latency period (Old et al., 1962, Ann. N.Y. Acad. Sci. 101:80-106; Bartlett, 1972, J. Natl. Cancer. Inst. 49:493-504). It has been demonstrated that T cell-mediated immunity is of critical importance for rejection of virally and chemically induced tumors (Klein et al., 1960, Cancer Res. 20:1561-1572; Tevethia et al., 1974, J. Immunol. 13:1417-1423).

Adoptive immunotherapy for tumors refers to the therapeutic approach wherein immune cells with antitumor activity are administered to a tumor-bearing host, with the objective that the cells cause the regression of an established tumor, either directly or indirectly. Immunization of hosts bearing established tumors with tumor cells or tumor antigens, as well a spontaneous tumors, has often been ineffective since the tumor may have already elicited an immunosuppressive response (Greenberg, 1987, Chapter 14, in Basic and Clinical Immunology, 6th ed., ed. by Stites, Stobo and Wells, Appleton and Lange, pp. 186-196; Bruggen, 1993). Thus, prior to immunotherapy, it had been necessary to reduce the tumor mass and deplete all the T cells in the tumor-bearing host (Greenberg et al., 1983, page 301-335, in “Basic and Clinical Tumor Immunology”, ed. Herbermann R R, Martinus Nijhoff).

Animal models have been developed in which hosts bearing advanced tumors can be treated by the transfer of tumor-specific syngeneic T cells (Mule et al., 1984, Science 225:1487-1489). Investigators at the National Cancer Institute (NCI) have used autologous reinfusion of peripheral blood lymphocytes or tumor-infiltrating lymphocytes (TIL), T cell cultures from biopsies of subcutaneous lymph nodules, to treat several human cancers (Rosenberg, S. A., U.S. Pat. No. 4,690,914, issued Sep. 1, 1987; Rosenberg et al., 1988, N. Engl. J. Med., 319:1676-1680). For example, TIL expanded in vitro in the presence of IL-2 have been adoptively transferred to cancer patients, resulting in tumor regression in select patients with metastatic melanoma. Melanoma TIL grown in IL-2 have been identified as CD3⁺ activated T lymphocytes, which are predominantly CD8⁺ cells with unique in vitro anti-tumor properties. Many long-term melanoma TIL cultures lyse autologous tumors in a specific class I MHC— and T cell antigen receptor-dependent manner (Topalian et al., 1989, J. Immunol. 142:3714).

Application of these methods for treatment of human cancers would entail isolating a specific set of tumor-reactive lymphocytes present in a patient, expanding these cells to large numbers in vitro, and then putting these cells back into the host by multiple infusions. Since T cells expanded in the presence of IL-2 are dependent upon IL-2 for survival, infusion of IL-2 after cell transfer prolongs the survival and augments the therapeutic efficacy of cultured T cells (Rosenberg et al., 1987, N. Engl. J. Med. 316:889-897). However, the toxicity of the high-dose IL-2 and activated lymphocyte treatment has been considerable, including high fevers, hypotension, damage to the endothelial wall due to capillary leak syndrome, and various adverse cardiac events such as arrhythmia and myocardial infarction (Rosenberg et al., 1988, N. Engl. J. Med. 319:1676-1680). Furthermore, the demanding technical expertise required to generate TILs, the quantity of material needed, and the severe adverse side effects limit the use of these techniques to specialized treatment centers.

CTLs specific for class I MHC-peptide complexes could be used in treatment of cancer and viral infections, and ways have been sought to generate them in vitro without the requirement for priming in vivo. These include the use of dendritic cells pulsed with appropriate antigens (Inaba et al., 1987, J. Exp. Med. 166:182-194; Macatonia et al., 1989, J. Exp. Med. 169:1255-1264; De Bruijn et al., 1992, Eur. J. Immunol. 22:3013-3020). RMA-S cells (mutant cells expressing high numbers of empty cell surface class I MHC molecules) loaded with peptide (De Bruijn et al., 1991, Eur. J. Immunol. 21:2963-2970; De Bruijn et al., 1992, supra; Houbiers et al., 1993, Eur. J. Immunol. 26:2072-2077) and macrophage phagocytosed-peptide loaded beads (De Bruijn et al., 1995, Eur. J. Immunol. 25, 1274-1285).

2.3 Dendritic Cells and Induction of Cancer Immunity

Dendritic cells are immunocytes classified as specialized antigen presenting cells. They are distributed throughout the body, especially subcutaneously. When bacteria, viruses, or foreign bodies, dendritic cells convey the information about the antigenicity of the bacterium, virus, or foreign body to lymphocytes and instruct lymphocytes to recognize the antigenicity and to react to it. Thus, dendritic cells play an important role at the earliest stage in causing the body to react immunologically. Cancer cells also have their own specific antigenicity, which can be recognized as a foreign body to the organism such as bacteria and viruses. However, cancer cells which arise and proliferate in the patient's body produce substances which inhibit such action of dendritic cells. Cancer cells are so structured as not to be killed by immunity.

Fusion of B cells or dendritic cells with tumor cells has been previously demonstrated to elicit anti-tumor immune responses in animal models (Guo et al., 1994, Science, 263:518-520; Stuhler and Walden, 1994, Cancer Immunol. Immuntother. 1994, 39:342-345; Gong et al., 1997, Nat. Med. 3:558-561; Celluzzi, 1998, J. Immunol. 160:3081-3085; Gong, PCT publication WO 98/46785, dated Oct. 23, 1998). In particular, immunization with hybrids of tumor cells and antigen presenting cells has been shown to result in protective immunity in various rodent models. Fused cells have functions of two kinds of cells: the function of cancer cells to produce cancer antigen and the function of dendritic cells to elicit an immune response.

However, the current treatments, while stimulating protective immunity, may not effectively treat a patient who already has an established disease. In other words, administration of fusion cells to a subject with cancer does not always stimulate an immune response sufficient to eliminate the disease. Thus, a need exists for a therapeutic composition which can be used to treat, e.g., cause the regression of an existing disease, e.g., cancer or infectious disease, in a patient.

Citation or discussion of a reference herein shall not be construed as an admission that such is prior art to the present invention.

### 3. SUMMARY OF THE INVENTION

The present invention provides methods and compositions for eliciting tumor-specific immunity in a subject by administering fusion cells comprising dendritic cells and tumor cells, together with recombinant human interleukin-12 (rhIL-12).

The present invention relates to methods and protocols for treating cancer using fusion cells formed by fusion of autologous dendritic cells and autologous non-dendritic cells administered in combination with a molecule which stimulates a CTL and/or humoral immune response. The invention is based, in part, on the discovery and demonstration that fusion cells of autologous dendritic cells (DCs) and autologous non-dendritic cells, e.g., tumor cells, when administered in combination with a molecule which stimulates a CTL and/or humoral immune response, results in a potentiated immune response against cancer. Such fusion cells combine the vigorous immunostimulatory effect of DCs with the specific antigenicity of tumor cells, thereby eliciting a specific and vigorous immune response. When autologous cells are used to prepare fusion cells, co-administration of the immune activator IL-12, enhances stimulation of the CTL and/or a humoral response.

The present invention further provides therapeutic methods by which dendritic cells are removed from a patient, treated with a cancer antigen ex vivo, and then returned into circulation of the patient together with recombinant human IL-12 (rhIL-12).

The present invention provides methods for administering fusion cells in combination with recombinant human interleukin-12. In particular, the invention provides specific regimens and dosages for administration of fusion cells and recombinant human interleukin-12. The present invention further provides specific methods for the generation of the fusion cells, and the treatment of the fusion cells before administering the fusion cells to the subject.

### 4. BRIEF DESCRIPTION OF THE FIGURES

FIGS. 1A-C. Fluorescence activated cell sorter (FACS) analysis of FCs. (A) DCs were stained by FITC-labeled anti-CD 80 antibody. A total of 34% of DCs were stained with anti-CD80 monoclonal antibody. (B) PKH26 was incorporated into glioma cells. More than 95% of glioma cells were positive for PKH26. (C) After incorporation of PKH26 into glioma cells, DCs and glioma cells were fused. DCs were stained with FITClabeled anti-CD80 monoclonal antibody. A total of 39.9% of cells were positive for both PKH26 and CD80, suggesting that most DCs were fused with glioma cells.

FIGS. 2A-B. Antitumor effects of immunization with FCs. (A) FCs (▴), DCs (▴), or irradiated parental cells as a control (●) were injected into syngeneic mice subcutaneously on days 0 and 7 (n=11 in each group). On day 14,1×106 parental cells were subcutaneously inoculated into the flank. The inoculated tumor cells caused large tumors within two weeks in all mice injected with irradiated parental cells. In contrast, none of the mice immunized with FCs died within six weeks. Whereas six of 11 mice immunized with DCs developed a palpable tumor that subsequently grew, none of 11 mice immunized with FCs developed a palpable tumor. (B) After immunization with FCs on days 0 and 7, 1×10⁴ tumor cells were stereotactically inoculated into the right frontal lobe of the brain (day 14). Half of the mice immunized with FCs survived longer than 70 days (▴; n=20 in each group; p<0.001) (FIG. 2-B). All control mice died within 6 weeks (●).

FIG. 3. Survival of mice following treatment with FCs and rIL-12. Parental cells (1×10⁴) were stereotactically inoculated into the right frontal lobe (day 0). On days 5 and 12, 3×10⁵ FCs were subcutaneously inoculated. Several mice were given an intraperitoneal (i.p.) injection of 0.5 pg/100 μl of rmlL-12, or 100 μl of saline, every other day for two weeks (3.5 pg/mouse total) starting on Day 5 and observed for 70 days. While vaccination with FCs alone did not prolong the survival of tumor-bearing mice (▴; p>0.05), vaccination with both FCs and rIL-12 prolonged the survival compared with the control (Δ; p=0.01). Five of ten mice treated with FCs and rIL-12 survived over seventy days.

FIG. 4. Cytotoxicity of spleen cells from tumor-bearing mice. SPCs were separated from untreated mice (●), mice injected with rIL-12 alone (Δ), mice injected DCs twice (days 0 and 7; ▴), mice immunized with FCs once (day 0; ◯) or twice (days 0 and 7;▪) and mice immunized with rIL-12 and FCs twice (days 0 and 7□;) on day 28. CTL activity on tumor cells from immunized mice, especially mice injected with rIL-12 and immunized with FCs twice, was considerably increased compared with the control and others. Antitumor activity on Yac-1 cell from treated mice increased but not considerably compared with the control (data not shown).

FIG. 5. Regression of established subcutaneous tumors following vaccination with FCs and depletion of T-cell subsets. Lymphocyte subsets were depleted by administering anti-CD4 (Δ), anti-CD8 (▴), anti-asialo GM1 (◯), or control rat IgG (▪) into mice given injections of glioma cells and FCs. On days 0 and 7, FCs were subcutaneously inoculated into the flank. Subsequently parental cells were inoculated into the opposite flank on day 14. The mAbs were injected i.p. on days 7, 10, 14, and 17. The antitumor effect was reduced in mice depleted of CD8⁺ T cells (▴) (n=4 in each group). The protection conferred by FCs was not abolished by CD4⁺ T and NK cell depletion. Control mice were not vaccinated with FCs (◯). Data represent means±SD.

FIGS. 6A-D. Immunofluorescence analysis of the developed brain tumors. A few CD4⁺ and CD8⁺ T cells were present in the tumors of non-vaccinated mice (FIGS. 6A, B). In contrast, many CD4⁺ and CD8⁺ T cells were seen in the tumors of vaccinated mice (FIGS. 6C, D). The numbers of infiltrating CD4⁺ and CD8⁺ T cells were almost the same. SR-B10.A cells were positive for GFAP.

FIG. 7. Fused cells stained with both FITC (green) and PKH-26 (red) among the PEG-treated cells FIG. 8. FACS analysis, cells stained with both PKH-2GL and PKH-26, which were considered to be fusions of DCs and BNL cells, are shown in upper area of cell scattergram with high forward scatter and high side scatter. The cell fraction of high and moderate forward scatter and low side scatter contained many non-fused BNL cells, which those of low forward scatter and low side scatter contained non-fused DCs and non-fused BNL cells. About 30% of the nonadherent cells were fusions as judged from the width of area of double positive cells occupying in the whole scattergram.

FIG. 9. FACS analysis of the cell fractions positive for both PKH-2GL and PKH26 gated on scattergram and examined for antigen expression. I-A^{d}/I-E^{d} (MCH class II), CD80, CD86 and CD54 molecules, which are found on DCs, were expressed by the fusions

FIG. 10. Scanning Electron Microscopy of BNL cells expressing short processes on a plain cell surface, whereas DCs have many long dendritic processes. The nonadherent fusion cells are large and ovoid with short dendritic processes.

FIG. 11. Vaccination of mice with DC/BNL fusions resulted in the rejection of a challenge with BNL cells inoculated in BALB/c mice. By contrast, injection of only DCs or only irradiated BNL cells failed to prevent the development and growth of tumors.

FIG. 12. Chromium-51 release assay of CTL. The effect of treatment with DC/BNL fusion cells alone against BNL tumor was not significant. However, injection of DC/BNL fusions followed by administration of IL-12 elicited a significant antitumor effect.

FIG. 13. Significant cytolytic activity against BNL cells was observed using splenocytes derived from mice treated with DC/BNL fusions. The solid bars are the BNL-cells and the hatched bars are the C26-cells.

FIG. 14. Splenocytes from mice treated with DC/BNL fusions in combination with IL-12 showed greater cytolytic activity against BNL cells than those treated with DC/BNL fusions alone.

FIG. 15. Lytic activity of the splenocytes treated with antibody against CD4 was significantly reduced, while those treated with antibody against CD8 exhibited almost the same lytic activity as those treated with an isotype identical antibody, rat IgG₂ₐ.

### 5. DETAILED DESCRIPTION OF THE INVENTION

The invention provides methods and compositions for the treatment of cancer. In a preferred embodiment, the methods of the invention provide the administration of fusion cells in combination with interleukin-12 (IL-12), e.g., recombinant human interleukin-12 (rhIL-12). The fusion cells of the invention are produced by fusion of autologous dendritic cells with autologous non-dendritic cells. Subsequently, the fused cells are administered to a subject in need thereof, in combination with a therapeutically effective dose of a molecule which stimulates a cytotoxic T-lymphocyte (CTL) response. In a preferred embodiment, the invention relates to methods and compositions for treating cancer comprising a therapeutically effective dose of fusion cells in combination with IL-12.

Using the methods described herein, autologous dendritic cells can be fused to a non-dendritic cell containing an antigen of interest, such as a cancer antigen. The resulting hybrids of dendritic cells and non-dendritic cells can be used as a potent composition against a disease condition involving an antigen, such as a cancer. This approach is particularly advantageous when a specific antigen is not readily identifiable, as in the case of many cancers. For treatment of human cancer, for example, non-dendritic cells can be obtained directly from the tumor of a patient. Fusion cell compositions prepared in this way are highly specific for the individual tumor being treated.

Described in the sections below are compositions and methods relating to such immunotherapeutic compositions. In particular, Sections 5.1, 5.2, and 5.3 describe the non-dendritic, dendritic, and the fusion cells, respectively, that are used with in the invention, and methods for their isolation, preparation, and/or generation. Target cancers that can be treated or prevented using such compositions are described below in Section 5.6. Sections 5.8, 5.9, and 5.10 describes the methods and use of these fusion cells as therapeutic compositions against cancer.

5.1 Non-Dendritic Cells

A non-dendritic cell of the present invention can be any cell bearing an antigen of interest for use in a fusion cell-cytokine composition. Such non-dendritic cells may be isolated from a variety of desired subjects, such as a tumor of a cancer subject. The nondendritic cells may also be from an established cell line or a primary cell culture. The methods for isolation and preparation of the non-dendritic cells are described in detail hereinbelow.

The source of the non-dendritic cells may be selected, depending on the nature of the disease with which the antigen is associated. Preferably, the non-dendritic cells are autologous to the subject being treated, i.e., the cells used are obtained from cells of the ultimate target tumor in vivo (e.g., tumor cells of the patient being treated), however, any non-dendritic cell can be used as long as at least one antigen present on the cell is an antigen specific to a cell obtained from the target tumor, and as long as the non-dendritic cell has the same class I MHC haplotype as the patient being treated. Thus, while whole cancer cells or other non-dendritic cells may be used in the present methods, it is not necessary to isolate them, or characterize or even know the identities of their antigens prior to performing the present methods.

For treatment or prevention of cancer, the non-dendritic cell is a cancer cell. In this embodiment, the invention provides fusion cells that express antigens expressed by cancer cells, e.g., tumor-specific antigens and tumor associated antigens, and are capable of eliciting an immune response against such cancer cells. In one embodiment of the invention, any tissues, or cells isolated from a cancer, including cancer that has metastasized to multiple sites, can be used for the preparation of non-dendritic cells. For example, leukemic cells circulating in blood, lymph or other body fluids can also be used, solid tumor tissue (e.g., primary tissue from a biopsy) can be used. Examples of cancers that are amenable to the methods of the invention are listed in Section 5.6 infra.

In a preferred embodiment, the tumor cells are not freshly isolated, but are instead cultured to select for tumor cells to be fused with dendritic cells and prevent or limit contamination of cells to be fused with healthy, non-cancerous or uninfected cells.

In a preferred embodiment, the non-dendritic cells of the invention may be isolated from a tumor that is surgically removed from mammal to be the recipient of the hybrid cell compositions. Prior to use, solid cancer tissue or aggregated cancer cells should be dispersed, preferably mechanically, into a single cell suspension by standard techniques. Enzymes, such as but not limited to, collagenase and DNase may also be used to disperse cancer cells. In yet another preferred embodiment, the non-dendritic cells of the invention are obtained from primary cell cultures, i.e., cultures of original cells obtained from the body. Typically, approximately 1×10⁶ to 1×10⁹ non-dendritic cells are used for formation of fusion cells.

In one embodiment, approximately 1×10⁶ to 1×10⁹ non-dendritic cells are used for formation of fusion cells. In another embodiment, 5×10⁷ to 2×10⁸ cells are used. In yet another embodiment, 5×10⁷ non-dendritic cells are used.

Cell lines derived from cancer or infected cells or tissues can also be used as non-dendritic cells, provided that the cells of the cell line have the same antigenic determinant(s) as the antigen of interest on the non-dendritic cells. Cancer or infected tissues, cells, or cell lines of human origin are preferred.

In an alternative embodiment, in order to prepare suitable non-dendritic cells that are cancer cells, noncancerous cells, preferably of the same cell type as the cancer desired to be inhibited can be isolated from the recipient or, less preferably, other individual who shares at least one MHC allele with the intended recipient, and treated with agents that cause the particular or a similar cancer or a transformed state; such agents may include but not limited to, radiation, chemical carcinogens, and viruses. Standard techniques can be used to treat the cells and propagate the cancer or transformed cells so produced.

Alternatively, if the gene encoding a tumor-specific antigen, tumor-associated antigen or antigen of the pathogen is available, normal cells of the appropriate cell type from the intended recipient. Optionally, more than one such antigen may be expressed in the recipient's cell in this fashion, as will be appreciated by those skilled in the art, any techniques known, such as those described in Ausubel et al. (eds., 1989, Current Protocols in Molecular Biology, Greene Publishing Associates and Wiley Interscience, New York), may be used to perform the transformation or transfection and subsequent recombinant expression of the antigen gene in recipient's cells. These non-dendritic cells bearing one or more MHC molecules in common with the recipient are suitable for use in the methods for formation of fusion cells of the invention.

The non-dendritic cells used for the generation of fusion cells and the target tumor or pathogen infected cell must have at least one common MHC allele in order to elicit an immune response in the mammal. Most preferred is where the non-dendritic cells are derived from the intended recipient (i.e., are autologous). Less preferred, the non-dendritic cells are nonautologous, but share at least one MHC allele with the cancer cells of the recipient. If the non-dendritic cells are obtained from the same or syngeneic individual, such cells will all have the same class I MHC haplotype. If they are not all obtained from the same subject, the MHC haplotype can be determined by standard HLA typing techniques well known in the art, such as serological tests and DNA analysis of the MHC loci. An MHC haplotype determination does not need to be undertaken prior to carrying out the procedure for generation of the fusion cells of the invention.

Non-dendritic cells, such as cells containing an antigen having the antigenicity of a cancer cell, can be identified and isolated by any method known in the art. For example, cancer or infected cells can be identified by morphology, enzyme assays, proliferation assays, or the presence of cancer-causing viruses. If the characteristics of the antigen of interest are known, non-dendritic cells can also be identified or isolated by any biochemical or immunological methods known in the art. For example, cancer cells or infected cells can be isolated by surgery, endoscopy, other biopsy techniques, affinity chromatography, and fluorescence activated cell sorting (e.g., with fluorescently tagged antibody against an antigen expressed by the cells).

There is no requirement that a clonal or homogeneous or purified population of non-dendritic cells be used. A mixture of cells can be used provided that a substantial number of cells in the mixture contain the antigen or antigens present on the tumor cells being targeted. In a specific embodiment, the non-dendritic cells and/or dendritic cells are purified.

In a specific embodiment, cancer tissue from the subject to be treated is collected during operation or by biopsy. The collected tissue is maintained in such a way as to keep the cancer cells alive. Cancer cells are preferably collected just prior preparation of the fusion cells. Most preferably, they are collected from ascites and pleural fluid just prior to preparation of fusion cells. When it is not possible to obtain sufficient quantities of malignant tumor cells in this manner, collection of malignant tumor cells from abdominal or thoracic liquid, or from a needle biopsy, may be possible.

5.2 Dendritic Cells

Dendritic cells can be isolated or generated from blood or bone marrow, or secondary lymphoid organs of the subject, such as but not limited to spleen, lymph nodes, tonsils, Peyer's patch of the intestine, and bone marrow, by any of the methods known in the art. Preferably, DCs used in the methods of the invention are (or terminally differentiated) dendritic cells. The source of dendritic cells is preferably human blood monocytes.

Immune cells obtained from such sources typically comprise predominantly recirculating lymphocytes and macrophages at various stages of differentiation and maturation. Dendritic cell preparations can be enriched by standard techniques (see e.g., Current Protocols in Immunology, 7.32.1-7.32.16, John Wiley and Sons, Inc., 1997). In one embodiment, for example, DCs may be enriched by depletion of T cells and adherent cells, followed by density gradient centrifugation. DCs may optionally be further purified by sorting of fuorescence-labeled cells, or by using anti-CD83 MAb magnetic beads.

Alternatively, a high yield of a relatively homogenous population of DCs can be obtained by treating DC progenitors present in blood samples or bone marrow with cytokines, such as granulocyte-macrophage colony stimulating factor (GM-CSF) and interleukin 4 (IL-4). Under such conditions, monocytes differentiate into dendritic cells without cell proliferation. Further treatment with agents such as TNFα stimulates terminal differentiation of DCs.

By way of example but not limitation, dendritic cells can be obtained from blood monocytes as follows: peripheral blood monocytes are obtained by standard methods (see, e.g., Sallusto et al., 1994, J. Exp. Med. 179:1109-1118). Leukocytes from healthy blood donors are collected by leukapheresis pack or buffy coat preparation using Ficoll-Paque density gradient centrifugation and plastic adherence. If mature DCs are desired, the following protocol may be used to culture DCs. Cells are allowed to adhere to plastic dishes for 4 hours at 37° C. Nonadherent cells are removed and adherent monocytes are cultured for 7 days in culture media containing 0.11 g/ml granulocyte-monocyte colony stimulating factor (GM-CSF) and 0.05 μg/ml interleukin-4 (IL-4). In order to prepare mature dendritic cells, tumor necrosis factor-α is added on day 5, and cells are collected on day 7.

In a specific embodiment, the following protocol is used. First, bone marrow is isolated and red cells lysed with ammonium chloride (Sigma, St. Louis, Mo.). Lymphocytes, granulocytes and DCs are depleted from the bone marrow cells and the remaining cells are plated in 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 pg/ml streptomycin, 10 ng/ml recombinant murine granulocyte-macrophage colony stimulating factor (GM-CSF; Becton Dickinson, San Jose, Calif.) and 30 U/ml recombinant mouse interleukin-4 (IL-4; Becton Dickinson). Second, on day 5 of culture, nonadherent and loosely adherent cells are collected and replated on 100 mm petri dishes (1×10 ⁶ cells/ml; 10 ml/dish). Next, GM-CSF and IL-4 in RPMI medium are added to the cells and 1×10⁶ DCs are mixed with 3×10⁶ irradiated (50 Gy, Hitachi MBR-1520R, dose rate: 1.1 Gy/min.) SR-B10.A cells. After 48 h, cells are collected for fusion with tumor cells.

Dendritic cells obtained in this way characteristically express the cell surface marker CD83. In addition, such cells characteristically express high levels of MHC class II molecules, as well as cell surface markers CD1α, CD40, CD86, CD54, and CD80, but lose expression of CD 14. Other cell surface markers characteristically include the T cell markers CD2 and CD5, the B cell marker CD7 and the myeloid cell markers CD13, CD32 (FcyR II), CD33, CD36, and CD63, as well as a large number of leukocyte-associated antigens Optionally, standard techniques, such as morphological observation and immunochemical staining, can be used to verify the presence of dendritic cells. For example, the purity of dendritic cells can be assessed by flow cytometry using fluorochrome-labeled antibodies directed against one or more of the characteristic cell surface markers noted above, e.g., CD83, HLA-ABC, HLA-DR, CD1α, CD40, and/or CD54. This technique can also be used to distinguish between immature and mature DCs, using fluorochrome-labeled antibodies directed against CD14, which is present in immature, but not mature, DCs.

In a preferred embodiment, venous blood is collected from the brachial vein by any method well-known to the skilled artisan. In a specific embodiment, 60 ml of blood is collected from the subject to be treated. White blood cells are separated from the collected blood, and only white blood cells with high adherent capacity are collected (see, e.g., Kikuchi et al., 2001, Cancer Immunol Immunother 50:337-344). An exemplary protocol for the cultivation of white blood cells with high adherent capacity is as follows. Briefly, peripheral blood mononuclear cells are separated from peripheral blood using Ficoll-25 Hypaque density centrifugation. Peripheral blood mononuclear cells are resuspended in RPMI-1640 (Sigma) and allowed to adhere to 24-well cluster plates. The nonadherent cells are removed after 2 hours at 37° C., and the adherent cells are subsequently cultured for 7 days in X-VIVO-15 medium (BioWhittaker, Walkersville, Md.) supplemented with 1% heat-inactivated autologous serum, 10 ng/ml recombinant human granulocyte-macrophage 30 colony stimulating factor (GM-CSF; Becton Dickinson, San Jose, Calif.), 30 U/ml recombinant human interleukin-4 (IL-4; Becton Dickinson), and 20 ng/ml tumor necrosis factor-α (TNF-α; Becton Dickinson). The cultures are fed every third day and are split when necessary. Thereafter, the semi-adherent and non-adherent cells are harvested by vigorous pipetting and used as dendritic cells for fusion. In certain embodiments, 50 mM 235 mercaptoethanol, 2 mM glutamate, 100 U/ml penicillin, and 100 mg/ml streptomycin are also present in the culture medium. GM-CSF and IL-4 cause white blood cells and lymphocytes to proliferate or to exhibit various functions. While culturing, serum of the appropriate subject is added to a concentration of 10% in the culture solution, avoiding any contact with heterogenous antigen.

In certain embodiments, the adherent cells are cultured in medium supplemented with at least 10 ng/ml, 20 ng/ml, 30 ng/ml, 40 ng/ml, 50 ng/ml, 60 ng/ml, 70 ng/ml, 80 ng/ml, 90 ng/ml or 100 ng/ml GM-CSF. In certain embodiments, the adherent cells are cultured in medium supplemented with at most 10 ng/ml, 20 ng/ml, 30 ng/ml, 40 ng/ml, 50 ng/ml, 60 ng/ml, 70 ng/ml, 80 ng/ml, 90 ng/ml or 100 ng/ml GM-CSF. In certain embodiments, the adherent cells are cultured in medium supplemented with between 10 ng/ml and 100 ng/ml, 20 ng/ml and 80 ng/ml, 30 ng/ml and 70 ng/ml, or 40 ng/ml and 60 ng/ml GM-C SF.

In certain embodiments, the adherent cells are cultured in medium supplemented with at least 10 ng/ml, 20 ng/ml, 30 ng/ml, 40 ng/ml, 50 ng/ml, 60 ng/ml, 70 ng/ml, 80 ng/ml, 90 ng/ml or 100 ng/ml TNF-α. In certain embodiments, the adherent cells are cultured in medium supplemented with at most 10 ng/ml, 20 ng/ml, 30 ng/ml, 40 ng/ml, 50 ng/ml, 60 ng/ml, 70 ng/ml, 80 ng/ml, 90 ng/ml or 100 ng/ml TNF-α. In certain embodiments, the adherent cells are cultured in medium supplemented with between 10 ng/ml and 100 ng/ml, 20 ng/ml and 80 ng/ml, 30 ng/ml and 70 ng/ml, or 40 ng/ml and 60 ng/ml TNF-α.

In certain embodiments, the adherent cells are cultured in medium supplemented with at least 10 U/ml, 20 U/ml, 30 ng/ml, 40 U/ml, 50 U/ml, 60 U/ml, 70 U/ml, 80 U/ml, 90 U/ml or 100 U/ml IL-4. In certain embodiments, the adherent cells are cultured in medium supplemented with at most 10 U/ml, 20 U/ml, 30 U/ml, 40 U/ml, 50 U/ml, 60 U/ml, 70 U/ml, 80 U/ml, 90 U/ml or 100 U/ml IL-4. In certain embodiments, the adherent cells are cultured in medium supplemented with between 10 U/ml and 100 U/ml, 20 U/ml and 80 U/ml, 30 U/ml and 70 U/ml, or 40 U/ml and 60 U/ml IL-4.

5.3 Generation of Fusion Cells

Non-dendritic cells can be fused to autologous DCs as followed. Cells can be washed prior to fusion under sterile conditions. Fusion can be accomplished by any cell fusion technique in the art provided that the fusion technique results in a mixture of fused cells suitable for injection into a mammal for treatment of cancer. In a specific example, electrofusion is used. Electrofusion techniques are well known in the art (Stuhler and Walden, 1994, Cancer Immunol. Immunother. 39: 342-345; see Chang et al. (eds.), Guide to Electroporation and Electrofusion. Academic Press, San Diego, 1992).

In a specific embodiment, the following protocol is used. In the first step, approximately 5×10⁷ tumor cells and 5×10⁷ dendritic cells (DCs) are suspended in 0.3 M glucose and transferred into an electrofusion cuvette. The sample is dielectrophoretically aligned to form cell-cell conjugates by pulsing the cell sample at 100 V/cm for 5-10 secs. Optionally, alignment may be optimized by applying a drop of dielectrical wax onto one aspect of the electroporation cuvette to ‘inhomogenize’ the electric field, thus directing the cells to the area of the highest field strength. In a second step, a fusion pulse is applied. Various parameters may be used for the electrofusion. For example, in one embodiment, the fusion pulse may be from a single to a triple pulse. In another embodiment, electrofusion is accomplished using from 500 to 1500 V/cm, preferably, 1,200V/cm at about 25 μF.

In a preferred embodiment, matured dendritic cells are fused with cancer cells by use of polyethyleneglycol. Briefly, the dendritic cells are mixed with lethally irradiated cancer cells (300 Gy, Hitachi MBR-1520R, dose rate 1.1 Gy/min). In certain embodiments, the cancer cells are irradiated with 10 Gy, 25 Gy, 50 Gy, 100 Gy, 200 Gy, 300 Gy, 400 Gy, 500 Gy, 750 Gy, or 1,000 Gy. In certain embodiments, the cancer cells are irradiated with 50 to 500 Gy. The ratio of dendritic cells and cancer cells can range from 3:1 to 10:1 depending on the numbers of acquired dendritic cells and cancer cells. Subsequently, fusion is initiated by adding 500 μl of a 50% solution of polyethylene glycol (PEG; Sigma) dropwise for 60 seconds. The fusion is stopped by stepwise addition of serum-free RPMI medium. After washing 3 times with phosphate-buffered saline (PBS; Cosmo Bio), fusion cells are plated in 100-mm petri dishes in the presence of GM-CSF, IL-4, and TNF-α in RPMI medium for 24 hours. In a specific embodiment, fusion cells are plated in the presence of 10 ng/ml GM-CSF, 30 U/ml IL-4, and 20 ng/ml TNF-α in RPMI medium for 24 hours After overnight culture, the fused cells are suspended in about 1 mL of physiological saline, and injected subcutaneously to the subject. In a preferred embodiment the suspension of fusion cells is injected in the groin area as this area is rich in lymph nodes.

In another specific embodiment, the following protocol is used. First, dendritic cells are prepared, as described in Section 5.2, above. On day 5 of dendritic cell culture, nonadherent and loosely adherent cells are collected and replated on 100-mm petri dishes (1×10⁶ cells/ml; 10 ml/dish). Next, GM-CSF and IL-4 in RPMI medium are added to the cells and 1×10⁶ DCs are mixed with 3×10⁶ irradiated (50 Gy, Hitachi MBR-1520R, dose rate: 1.1 Gy/min.) SR-B10.A cells. After 48 h, fusion is started by adding dropwise for 60 sec, 500 μl of a 50% solution of polyethylene glycol (PEG; Sigma). In a specific embodiment, the final concentration of PEG is 2.5%. In certain embodiments of the invention, the final concentration of PEG is 0.5%, 1%, 1.5%, 2.5%, 5%, 10%, 15%, 20% or 25%. In certain embodiments, the final concentration of PEG is 0.5% to 25%, 1% to 20%, or 5% to 15%. The fusion is stopped by stepwise addition of serum-free RPMI medium. FCs are plated in 100-mm petri dishes in the presence of GM-CSF and IL-4 in RPMI medium for 48 h.

In another embodiment, the dendritic cell and the non-dendritic cell are fused as described above. Subsequently, the fused cells are transfected with genetic material which encodes a molecule which stimulates a CTL and/or humoral immune response. In a preferred embodiment, the genetic material is mRNA which encodes IL-12. Preferred methods of transfection include electroporation or cationic polymers.

In certain embodiments, the cancer cells are fused with the dendritic cells at a ratio of 1 cancer cell per dendritic cell (DC), 2 cancer cells per DC, 3 cancer cells per DC, 4 cancer cells per DC, 5 cancer cells per DC, 6 cancer cells per DC, 7 cancer cells per DC, 8 cancer cells per DC, 9 cancer cells per DC, or 10 cancer cells per DC.

The extent of fusion cell formation within a population of antigenic and dendritic cells can be determined by a number of diagnostic techniques known in the art. In one embodiment, for example, hybrids are characterized by emission of both colors after labeling of DCs and tumor cells with red and green intracellular fluorescent dyes, respectively. Samples of DCs without tumor cells, and tumor cells without DCs can be used as negative controls, as well as tumor+DC mixture without electrofusion.

In one embodiment, the fusion cells prepared by this method comprise approximately 10 and 20% of the total cell population. In yet another embodiment, the fusion cells prepared by this method comprise approximately 5 to 50% of the total cell population.

In certain embodiments, the fusion cells are cultured in medium supplemented with at least 10 ng/ml, 20 ng/ml, 30 ng/ml, 40 ng/ml, 50 ng/ml, 60 ng/ml, 70 ng/ml, 80 ng/ml, 90 ng/ml or 100 ng/ml GM-CSF. In certain embodiments, the adherent cells are cultured in medium supplemented with at most 10 ng/ml, 20 ng/ml, 30 ng/ml, 40 ng/ml, 50 ng/ml, 60 ng/ml, 70 ng/ml, 80 ng/ml, 90 ng/ml or 100 ng/ml GM-CSF. In certain embodiments, the adherent cells are cultured in medium supplemented with between 10 ng/ml and 100 ng/ml, 20 ng/ml and 80 ng/ml, 30 ng/ml and 70 ng/ml, or 40 ng/ml and 60 ng/ml GM-CSF.

In certain embodiments, the fusion cells are cultured in medium supplemented with at least 10 ng/ml, 20 ng/ml, 30 ng/ml, 40 ng/ml, 50 ng/ml, 60 ng/ml, 70 ng/ml, 80 ng/ml, 90 ng/ml or 100 ng/ml TNF-α. In certain embodiments, the adherent cells are cultured in medium supplemented with at most 10 ng/ml, 20 ng/ml, 30 ng/ml, 40 ng/ml, 50 ng/ml, 60 ng/ml, 70 ng/ml, 80 ng/ml, 90 ng/ml or 100 ng/ml TNF-α. In certain embodiments, the adherent cells are cultured in medium supplemented with between 10 ng/ml and 100 ng/ml, 20 ng/ml and 80 ng/ml, 30 ng/ml and 70 ng/ml, or 40 ng/ml and 60 ng/ml TNF-α.

In certain embodiments, the fusion cells are cultured in medium supplemented with at least 10 U/ml, 20 U/ml, 30 ng/ml, 40 U/ml, 50 U/ml, 60 U/ml, 70 U/ml, 80 U/ml, 90 U/ml or 100 U/ml IL-4. In certain embodiments, the adherent cells are cultured in medium supplemented with at most 10 U/ml, 20 U/ml, 30 U/ml, 40 U/ml, 50 U/ml, 60 U/ml, 70 U/ml, 80 U/ml, 90 U/ml or 100 U/ml IL-4. In certain embodiments, the adherent cells are cultured in medium supplemented with between 10 U/ml and 100 U/ml, 20 U/ml and 80 U/ml, 30 U/ml and 70 U/ml, or 40 U/ml and 60 U/ml IL-4.

To prevent contamination of the fusion cells with bacteria or viruses, cell culturing and fusion of cells may be conducted in a room for exclusive use for mammalian cell culturing. These cells are monitored to confirm they are not infected with bacteria or contaminated with the toxin of bacteria.

In instances where dendritic cells are fused with cancer cells by use of polyethylene glycol or another method, some, but not all of the cancer cells may be fused. When such non-fused cancer cells are injected into the subject's body, unfavorable effects may occur. Therefore, in certain embodiments, the cancer cells are irradiated before administration. By use of these safety measures, there is little possibility that cancer cells proliferate actively even if the fused cells are contaminated with a trace amount of cancer cells. In a preferred embodiment, the cancer cells are irradiated before fusion.

In certain embodiments, the cancer cells are obtained from a subject at least 10 min, 30 min, 60 min, 2 hours, 5 hours, 10 hours, or 24 hours before fusing the cancer cells with dendritic cells. In certain embodiments, the cancer cells are obtained from a subject at most 10 min, 30 min, 60 min, 2 hours, 5 hours, 10 hours, or 24 hours before fusing the cancer cells with dendritic cells. In certain embodiments, the cancer cells are obtained from a subject and subsequently a cell line is established before fusing the cancer cells with dendritic cells. In certain embodiments, the dendritic cells are obtained from a subject at least 10 min, 30 min, 60 min, 2 hours, 5 hours, 10 hours, or 24 hours before fusing the cancer cells with dendritic cells. In certain embodiments, the dendritic cells are obtained from a subject at most 10 min, 30 min, 60 min, 2 hours, 5 hours, 10 hours, or 24 hours before fusing the cancer cells with dendritic cells.

5.3.1 Recombinant Cells

In an alternative embodiment, rather than fusing a dendritic cell to a cancer cell, the non-dendritic cells are transfected with a gene encoding a known antigen of a cancer. The non-dendritic cells are then selected for those expressing the recombinant antigen and administered to the subject in need thereof in combination with a cytokine or molecule which stimulates or induces a CTL and/or humoral immune response.

Recombinant expression of a gene by gene transfer, or gene therapy, refers to the administration of a nucleic acid to a subject. The nucleic acid, either directly or indirectly via its encoded protein, mediates a therapeutic effect in the subject. The present invention provides methods of gene therapy wherein genetic material, e.g., DNA or mRNA, encoding a protein of therapeutic value (preferably to humans) is introduced into the fused cells according to the methods of the invention, such that the nucleic acid is expressible by the fused cells, followed by administration of the recombinant fused cells to a subject.

The recombinant fused cells of the present invention can be used in any of the methods for gene therapy available in the art. Thus, the nucleic acid introduced into the cells may encode any desired protein, e.g., an antigenic protein or portion thereof or a protein that stimulates a CTL and/or humoral immune response. The descriptions below are meant to be illustrative of such methods. It will be readily understood by those of skill in the art that the methods illustrated represent only a sample of all available methods of gene therapy.

For general reviews of the methods of gene therapy, see Lundstrom, 1999, J. Recept. Signal Transduct. Res. 19:673-686; Robbins and Ghivizzani, 1998, Pharmacol. Ther.80:35-47; Pelegrin et al., 1998, Hum. Gene Ther. 9:2165-2175; Harvey and Caskey, 1998, Curr. Opin. Chem. Biol. 2:512-518; Guntaka and Swamynathan, 1998, Indian J. Exp. Biol. 36:539-535; Desnick and Schuchman, 1998, Acta Paediatr. Jpn. 40:191-203; Vos, 1998, Curr. Opin. Genet. Dev. 8:351-359; Tarahovsky and Ivanitsky, 1998, Biochemistry (Mosc) 63:607-618; Morishita et al., 1998, Circ. Res. 2:1023-1028; Vile et al., 1998, Mol. Med. Today 4:84-92; Branch and Klotman,1998, Exp. Nephrol. 6:78-83; Ascenzioni et al., 1997, Cancer Lett. 118:135-142; Chan and Glazer, 1997, J. Mol. Med. 75:267-282. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In an embodiment in which recombinant cells are used in gene therapy, a gene whose expression is desired in a subject is introduced into the fused cells such that it is expressible by the cells and the recombinant cells are then administered in vivo for therapeutic effect.

Recombinant fused cells can be used in any appropriate method of gene therapy, as would be recognized by those in the art upon considering this disclosure. The resulting action of recombinant manipulated cells administered to a subject can, for example, lead to the activation or inhibition of a pre-selected gene, such as activation of IL-12, in the patient, thus leading to improvement of the diseased condition afflicting the patient.

The desired gene is transferred, via transfection, into fused by such methods as electroporation, lipofection, calcium phosphate mediated transfection, or viral infection. Usually, the method of transfer includes the transfer of a vector containing a selectable marker. The cells are then placed under selection to isolate those cells that have taken up and are expressing the vector, containing the selectable marker and also the transferred gene. Those cells are then delivered to a patient.

In this embodiment, the desired gene is introduced into fused, cells prior to administration in vivo of the resulting recombinant cell. Such introduction can be carried out by any method known in the art, including but not limited to transfection, electroporation, microinjection, infection with a viral or bacteriophage vector containing the gene sequences, cell fusion, chromosome-mediated gene transfer, microcell-mediated gene transfer, spheroplast fusion, elc. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the gene to the cell, so that the gene is expressible by the cell and preferably heritable and expressible by its cell progeny.

One common method of practicing gene therapy is by making use of retroviral vectors (see Miller et al., 1993, Meth. Enzymol. 217:581-599). A retroviral vector is a retrovirus that has been modified to incorporate a preselected gene in order to effect the expression of that gene. It has been found that many of the naturally occurring DNA sequences of retroviruses are dispensable in retroviral vectors. Only a small subset of the naturally occurring DNA sequences of retroviruses is necessary. In general, a retroviral vector must contain all of the cis-acting sequences necessary for the packaging and integration of the viral genome. These cis-acting sequences are:

a) a long terminal repeat (LTR), or portions thereof, at each end of the vector;

b) primer binding sites for negative and positive strand DNA synthesis; and

c) a packaging signal, necessary for the incorporation of genomic RNA into virions.

The gene to be used in gene therapy is cloned into the vector, which facilitates delivery of the gene into an cell by infection or delivery of the vector into the cell.

More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses can be used to deliver genes to non-dendritic cells derived from the liver, the central nervous system, endothelium, and muscle. Adenoviruses have the advantage of being capable of infecting non-dividing cells. Kozarsky and Wilson, 1993, Current Opinion in Genetics and Development 3:499-503 present a review of adenovirus-based gene therapy. Other instances of the use of adenoviruses in gene therapy can be found in Rosenfeld et al, 1991, Science 252:431-434; Rosenfeld et al., 1992, Cell 68:143-155; and Mastrangeli et al., 1993, J. Clin. Invest. 91:225-234.

It has been proposed that adeno-associated virus (AAV) be used in gene therapy (Walsh et al., 1993, Proc. Soc. Exp. Biol. Med. 204:289-300). It has also been proposed that alphaviruses be used in gene therapy (Lundstrom, 1999, J. Recept. Signal Transduct. Res. 19:673-686).

Other methods of gene delivery in gene therapy include mammalian artificial chromosomes (Vos, 1998, Curr. Op. Genet. Dev. 8:351-359); liposomes (Tarahovsky and Ivanitsky, 1998, Biochemistry (Mosc) 63:607-618); ribozymes (Branch and Klotman, 1998, Exp. Nephrol. 6:78-83); and triplex DNA (Chan and Glazer, 1997, J. Mol. Med. 75:267-282).

A desired gene can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, the desired gene recombinantly expressed in the cell to be introduced for purposes of gene therapy comprises an inducible promoter operably linked to the coding region, such that expression of the recombinant gene is controllable by controlling the presence or absence of the appropriate inducer of transcription.

In a preferred embodiment, the desired gene recombinantly expressed in the cells, whether its function is to elicit a cell fate change according to the methods of the invention, is flanked by Cre sites. When the gene function is no longer required, the cells comprising the recombinant gene are subjected to Lox protein, for example be means of supplying a nucleic acid containing the Lox coding sequences functionally coupled to an inducible or tissue specific promoter, or by supplying Lox protein functionally coupled to a nuclear internalization signal. Lox recombinase functions to recombine the Cre sequences (Hamilton et al., 1984, J. Mol. Biol. 178:481-486), excising the intervening sequences in the process, which according to this embodiment contain a nucleic acid of a desired gene. The method has been used successfully to manipulate recombinant gene expression (Fukushige et al, 1992, Proc. Natl. Acad. Sci. USA 89:7905-7909). Alternatively, the FLP/FRT recombination system can be used to control the presence and expression of genes through site-specific recombination (Brand and Perrimon, 1993, Development 118:401-415).

In a preferred aspect of the invention, gene therapy using nucleic acids encoding hepatitis B or hepatitis C major antigens are directed to the treatment of viral hepatitis.

5.4 Immune Cell Activating Molecules

The present invention provides a method which comprises administering first, a fusion cell derived from the fusion of a dendritic and non-dendritic cell, and second, a cytokine or other molecule which can stimulate or induce a cytotoxic T cell (CTL) response, such as interleukin-12 (IL-12).

IL-12 plays a major role in regulating the migration and proper selection of effector cells in an immune response. The IL-12 gene product polarizes the immune response toward the TH, subset of T helper cells and strongly stimulates CTL activity. In a preferred embodiment, the CTL stimulating molecule is IL-12. As elevated doses of IL-12 exhibits toxicity when administered systemically, IL-12 is preferably administered locally. Additional modes of administration are described below in Section 5.7.1.

Expression of IL-12 receptor β2 (IL-12R-β2) is necessary for maintaining IL-12 responsiveness and controlling TH₁, lineage commitment. Furthermore, IL-12 signaling results in STAT4 activation, i.e., measured by an increase of phosphorylation of STAT4, and interferon-γ (IFN-γ) production. Thus, in one embodiment, the present invention contemplates the use of a molecule, which is not IL-12, which can activate STAT4, for example a small molecule activator of STAT4 identified by the use of combinatorial chemistry.

In an alternative embodiment, the immune stimulating molecule is IL-18. In yet another embodiment, the immune stimulating molecule is IL-15. In yet another embodiment, the immune stimulating molecule is interferon-γ.

In another embodiment, the subject to be treated is given any combination of molecules or cytokines described herein which stimulate or induce a CTL and/or humoral immune response.

In a less preferred embodiment, to increase the cytotoxic T-cell pool, i.e., the TH, cell subpopulation, anti-IL-4 antibodies can be added to inhibit the polarization of T-helper cells into TH₂ cells, thereby creating selective pressure toward the TH₁ subset of T-helper cells. Further, anti-IL-4 antibodies can be administered concurrent with the administration of IL-12, to induce the TH cells to differentiate into TH₁ cells. After differentiation, cells can be washed, resuspended in, for example, buffered saline, and reintroduced into a subject via, preferably, intravenous administration.

The present invention also pertains to variants of the above-described interleukins. Such variants have an altered amino acid sequence which can function as agonists (mimetics) to promote a CTL and/or humoral immune response response. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a molecule capable of stimulating a CTL and/or humoral immune response can be identified by screening combinatorial libraries of mutants, e.g., truncation mutants, for agonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of IL-12 from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem., 53:323; Itakura et al., 1984, Science, 198:1056; Ike et al., 1983, Nucleic Acid Res., 11:477).

In addition, libraries of fragments of the coding sequence of an interleukin capable of promoting a CTL and/or humoral immune response can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of an interleukin capable of promoting a CTL and/or humoral immune response (Arkin and Yourvan, 1992, Proc. Natl. Acad. Sci. USA, 89:7811-7815; Delgrave et al., 1993, Protein Engineering, 6(3):327-331).

In a specific embodiment of the invention fusion cells are administered in combination with recombinant human interleukin-12. Recombinant human interleukin-12, previously called cytotoxic lymphocyte maturation factor (CLMF) or NK cell stimulatory factor (NKSF), is generated according to the methods provided in the following publications, which are incorporated by reference in their entireties herein: Stern et al., 1990, Proc Natl Acad Sci 87:6808-6812; Gubler et al., 1991, Proc Natl Acad Sci 88:4143-4147; and Wolf et al., 1991, J Immunol 146:3074-3081.

In a preferred embodiment of the invention, rhIL-12 is administered to the subject before the combined immunotherapy to determine whether the subject is hypersensitive to hIL-12. In a specific embodiment, hypersensitivity to hIL-12 is tested by injecting rhIL-12 subcutaneously. In specific embodiments, a prick-test is used to test whether the subject is hypersensitive to hIL-12. In specific embodiments, drops of solutions of different concentrations of hIL-12, e.g., 1 femtomole, 10 femtomole, 100 femtomole, 1 picomole, 10 picomole, 100 picomole, 1 nanomole, 10 nanomole, 100 nanomole, 1 micromole, 10 micromole, 100 micromole, 1 millimole, 10 millimole, or 100 millimole are applied to the skin of the subject's arm. The skin is pricked with a needle at the positions of the drops, and the reaction of the skin is observed over a time period of 5 minutes to 60 minutes. Redness of the skin and skin rash are indicators of a hypersensitive reaction. The severity of possible adverse reactions to and therapeutic efficacy of rhIL-12 in a subject are evaluated by conducting the following tests: hematological tests, urinalysis and fecal test, and imaging examinations including CT scan. The efficacy of rhIL-12 can be estimated by measuring tumor size in the subject. From animal experiments conducted heretofore, it has been shown that administration of interleukin-12 may be followed by reduction in size or disappearance of tumor implanted experimentally.

In certain embodiments, about 10 ng, 20 ng, 30 ng, 40 ng, 50 ng, 60 ng, 70 ng, 80 ng, 90 ng or 100 ng of interleukin-12 per kg of body weight are administered per administration. In certain embodiments, between 10 ng and 25 ng, 25 ng and 50 ng, 50 ng and 75 ng, 75 ng and 100 ng, 10 ng and 100 ng, or 25 ng and 75 ng of interleukin-12 per kg of body weight are administered per administration. In a preferred embodiment, 30 ng of interleukin-12 per kg of body weight are administered per administration.

5.5 Assays for Measuring an Immune Response

The fusion cell-cytokine compositions can be assayed for immunogenicity using any method known in the art. By way of example but not limitation, one of the following procedures can be used.

A humoral immune response can be measured using standard detection assays including but not limited to an ELISA, to determine the relative amount of antibodies which recognize the target antigen in the sera of a treated subject, relative to the amount of antibodies in untreated subjects. A CTL response can be measured using standard immunoassays including chromium release assays as described herein. More particularly, a CTL response is determined by the measurable difference in CTL activity upon administration a stimulator, relative to CTL activity in the absence of a stimulator.

5.5.1 MLTC Assay

The fusion cell-cytokine compositions may be tested for immunogenicity using a MLTC assay. For example, 1×10⁷ fusion cells are y-irradiated, and mixed with T lymphocytes. At various intervals the T lymphocytes are tested for cytotoxicity in a 4 hour ⁵¹Cr-release assay (see Palladino et al., 1987, Cancer Res. 47:5074-5079). In this assay, the mixed lymphocyte culture is added to a target cell suspension to give different effector:target (E:T) ratios (usually 1:1 to 40:1). The target cells are prelabelled by incubating 1×10⁶ target cells in culture medium containing 500 μCi ⁵¹Cr/ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelletted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm.

In order to block the MHC class I cascade a concentrated hybridoma supernatant derived from K-44 hybridoma cells (an anti-MHC class I hybridoma) is added to the test samples to a final concentration of 12.5%.

5.5.2 Antibody Response Assay

In one embodiment of the invention, the immunogenicity of fusion cells is determined by measuring antibodies produced in response to the vaccination, by an antibody response assay, such as an enzyme-linked immunosorbent assay (ELISA) assay. Methods for such assays are well known in the art (see, e.g., Section 2.1 of Current Protocols in Immunology, Coligan et al. (eds.), John Wiley and Sons, Inc. 1997). In one mode of the embodiment, microtitre plates (96-well Immuno Plate II, Nunc) are coated with 50 μl/well of a 0.75 pg/ml solution of a purified cancer cell or infected used in the composition in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient) is applied at 20° C. for 1 hour, and the plates are washed 3 times with PBS-T. The antigen antibody activity is then measured calorimetrically after incubating at 20° C. for 1 hour with 50 μl/well of sheep anti-mouse or anti-human immunoglobulin, as appropriate, conjugated with horseradish peroxidase diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a photometer at 492 nm (ref. 620 nm), using standard techniques.

5.5.3 Cytokine Detection Assays

The CD4⁺ T cell proliferative response to the fusion cell-cytokine composition may be measured by detection and quantitation of the levels of specific cytokines. In one embodiment, for example, intracellular cytokines may be measured using an IFN-γ detection assay to test for immunogenicity of the fusion cell-cytokine composition. In an example of this method, peripheral blood mononuclear cells from a subject treated with the fusion cell-cytokine composition are stimulated with peptide antigens such as mucin peptide antigens or Her2/neu derived epitopes. Cells are then stained with T cell-specific labeled antibodies detectable by flow cytometry, for example FITC-conjugated anti-CD8 and PerCP-labeled anti-CD4 antibodies. After washing, cells are fixed, permeabilized, and reacted with dye-labeled antibodies reactive with human IFN-γ (PE-anti-IFN-γ). Samples are analyzed by flow cytometry using standard techniques.

Alternatively, a filter immunoassay, the enzyme-linked immunospot assay (ELISPOT) assay, may be used to detect specifc cytokines surrounding a T cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of mononuclear blood cells, containing cytokine-secreting cells, obtained from a subject vaccinated with a fusion cell-cytokine composition, is diluted onto the wells of the microtitre plate. A labeled, e.g., biotin-labeled, secondary anti-cytokine antibody is added. The antibody cytokine complex can then be detected, i.e. by enzyme-conjugated streptavidin—cytokine-secreting cells will appear as “spots” by visual, microscopic, or electronic detection methods.

5.5.4 Tetramer Staining Assay

In another embodiment, the “tetramer staining” assay (Altman et al., 1996, Science 274: 94-96) may be used to identify antigen-specific T-cells. For example, in one embodiment, an MHC molecule containing a specific peptide antigen, such as a tumorspecific antigen, is multimerized to make soluble peptide tetramers and labeled, for example, by complexing to streptavidin. The MHC complex is then mixed with a population of T cells obtained from a subject treated with a fusion cell composition. Biotin is then used to stain T cells which express the antigen of interest, i.e., the tumor-specific antigen.

Cytotoxic T-cells are immune cells which are CD8 positive and have been activated by antigen presenting cells (APCs), which have processed and are displaying an antigen of a target cell. The antigen presentation, in conjunction with activation of co-stimulatory molecules such as B-7/CTLA-4 and CD40 leads to priming of the T-cell to target and destroy cells expressing the antigen.

Cytotoxic T-cells are generally characterized as expressing CD8 in addition to secreting TNF-β, perforin and IL-2. A cytotoxic T cell response can be measured in various assays, including but not limited to increased target cell lysis in ⁵¹Cr release assays using T-cells from treated subjects, in comparison to T-cells from untreated subjects, as shown in the examples herein, as well as measuring an increase in the levels of IFN-γ and IL-2 in treated subjects relative to untreated subjects.

5.6 Target Cancers

The cancers and oncogenic diseases that can be treated or prevented using the fusion cells of the invention of the present invention include, but are not limited to: human sarcomas and carcinomas, e.g., renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease.

5.7 Patient Selection and Possible Side Effects and Their Treatment

A variety of therapies have been developed for the treatment of cancer. The optimal treatment for individual patients should be chosen by taking into consideration the type and stage of cancers in individual patients. The basic selection criteria for patients to be enrolled in a clinical study involving combined immunotherapy are as follows:

In accordance with the present invention, the following inclusion criteria should be considered in order to select patients:

1. Patients who received existing established anticancer therapy with no satisfying or reliable effects, and whose lesions are being progressing, i.e., patients who received surgical operation, chemotherapy, radiotherapy and other established anticancer therapy with no satisfying or reliable effect and in whom the cancer is progressing or expected to progress in the future;

2. Patients for whom, for some reason, the existing anticancer therapies cannot be applied;

3. Patients for whom any of the existing anticancer therapy is not indicated due to the stage of advance of the lesion, but some efficacy or recovery in general condition to some extent or more can be expected from the treatment;

4. Patients in whom no severe immunodeficiency is present regardless of cause; and

5. Patients from whom a small amount of cancer tissues or cells can be collected safely and assuredly and it is feasible to draw 60 mL of blood sample at one time to collect dendritic cells.

In a preferred embodiment of the invention, the subject is continuously monitored for the occurrence of possible side effects of rhIL-12. The skilled practitioner will be aware of such potential side effects experienced by patients who have received single or multiple doses of rhIL-12, the most common of which effects fever, headache, nausea, chills, weakness and swelling, redness, irritation, itching and/or pain at injection site (for injections under the skin). Also, patients have experienced a temporary rise in blood sugar levels and liver enzymes and a temporary decrease in numbers of white blood cells.

Other less common side effects following doses of rhIL-12 include muscle and joint aches, sleeplessness, dizziness, stomach pain, diarrhea, vomiting, loss of appetite, sore throat, increased cough, runny nose, sweating, pain, general discomfort, constipation, mouth sores, and decrease in platelets (cells that help the blood clot) which may result in easy bruising or bleeding because of a decreased ability of the subject's blood to clot.

On rare occasions, after receiving multiple high doses of rhIL-12, patients have experienced anxiety, confusion, depression, blood in stool and vomit, failed kidney function, burning or tingling of the skin, shortness of breath, upset stomach, more acid in blood than normal, low blood pressure and high blood pressure. Deaths have occurred in cancer patients receiving high doses of rhIL-12 given multiple times intravenously (into the vein).

Animal reproductive studies have been performed with rhIL-12, and death of the fetus, abortion, and reduced fetal weights have occurred. These effects are consistent with those that occur with other compounds of this nature. No studies examining the effects of rhIL-12 on fertility have been performed to date. It is not known whether rhIL-12 is excreted in human milk. Since many drugs are excreted in milk, women who are nursing should not receive rhIL-12.

Mutagenicity studies have shown no effects of rhIL-12. No studies examining the cancer-causing potential of rhIL-12 have been performed.

Immediate severe reactions such as allergic reactions, shortness of breath, wheezing, and hives have not been observed in animal or human studies of rhIL-12. However, such reactions are possible after receiving any protein drug.

The subject receiving combined immunotherapy is instructed to tell the physician about any new health problems that develop. In a most preferred embodiment, the subject is monitored closely for these side effects. If symptoms develop, the skilled practitioner will reduce or withdraw therapy or initiate appropriate treatment. Other unexpected side effects that have not yet been previously observed may also occur.

The use of rhIL-12 poses possible risks to a fetus. If the subject is a woman of child bearing potential, the subject is required to have a pregnancy test (blood) done during the screening period. If the subject becomes pregnant during treatment with rhIL-12, the subject must tell the study physician. Because it is not known whether rhIL-12 is excreted in human milk, the subject must not nurse an infant or child while receiving rhIL-12.

The immunotherapeutic methods encompassed by the present invention may pose additional side effects. For example, lymphocytes release cytokines as part of the tumor-specific immune response, which may result in such symptoms such as fever, chill, discomfort, and hot feeling of the tumor site. These symptoms may be interpreted as inflammatory reactions in cancer tissues. When remarkable fever appears, treatment with antipyretics may be provided, as would be appreciated by the skilled practitioner. Antipyretics are substances capable of relieving or reducing fever and anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Fusion cells comprising dendritic and cancer cells are artificial cells and foreign to the patient being treated, they are not expected to survive in the patient after antigen is presented to the lymphocytes. In animal experiments, fused cells do not survive, and have not been observed to generate tumors. However, fusion cells for use in human immunotherapy should be irradiated to remove proliferating capacity before administering to the patient to prevent proliferation. Efficacy of fusion cells to induce cancer immunity in vivo is little affected by their irradiation.

The antigenicity of cancer cells may be largely overlapping with the antigenicity of normal cells of the subject receiving combined immunotherapy. Therefore, when cancer cells are killed immunologically, normal cells of the organs in which the cancer developed may also be injured by the same immunological effect, known as induction of autoimmune phenomenon. When dendritic cell immunotherapy and rhIL-12 therapy is combined, the immuno-reaction is expected to be enhanced. If such a phenomenon occurs in a subject receiving combined immunotherapy, it is possible that not only the cancerous tissues, but also normal tissues, are injured. Currently, there is no evidence that an autoimmune phenomenon powerful enough to damage normal tissues actually occurs in patients treated undergoing the immunotherapeutic methods described herein. However, results of animal experiments indicate that this point should be taken into consideration. If administration of immunosuppressants such as steroids is considered to be necessary based on the judgement of the skilled practitioner in view of the therapeutic efficacy for cancer and severity of damages to normal tissues, immunosuppressive therapy may be provided. Immunosuppressive agents are, inter alia, glucocorticoids (methylprednisolone), myelin basic protein (e.g., 7-capaxone), anti-Fc receptor monoclonal antibodies, hydroorotate dehydrogenase inhibitor, anti-IL2 monoclonal antibodies (e.g., CHI-621 and dacliximab), buspirone, castanospermine, CD-59 (complement factor inhibitor), 5-lipoxygenase inhibitor (e.g., CMI-392), phosphatidic acid synthesis antagonists, ebselen, edelfosine, enlimomab, galaptin, platelet activating factor antagonists, selectin antagonists (e.g., ICAM4), interleukin-10 agonist, macrocylic lactone, methoxatone, mizoribine, OX-19, peptigen agents, PG-27, protein kinase C inhibitors, phosphodiesterase IV inhibitor, single chain antigen binding proteins, complement factor inhibitor, sialophorin, sirolimus, spirocyclic lactams, 5-hydroxytryptamine antagonist, anti-TCR monoclonal antibodies, CD5 gelonin and TOK-8801.

5.8 Pharmaceutical Preparations and Methods of Administration

The composition formulations of the invention comprise an effective immunizing amount of the fusion cells which are to be administered with a molecule capable of stimulating a CTL and/or humoral immune response, e.g., cytokines.

Suitable preparations of fusion cell-cytokine compositions include injectables, preferably as a liquid solution.

Many methods may be used to introduce the composition formulations of the invention; these include but are not limited to subcutaneous injection, intralymphatically, intradermal, intramuscular, intravenous, and via scarification (scratching through the top layers of skin, e.g., using a bifurcated needle). In a specific embodiment, fusion cellcytokine compositions are injected intradermally.

In addition, if desired, the composition preparation may also include minor amounts of auxiliary substances such as wetting or emulsifying agents, pH buffering agents, and/or compounds which enhance the effectiveness of the composition. The effectiveness of an auxiliary substances may be determined by measuring the induction of antibodies directed against a fusion cell.

The mammal to which the composition is administered is preferably a human, but can also be a non-human animal including but not limited to cows, horses, sheep, pigs, fowl (e.g., chickens), goats, cats, dogs, hamsters, mice and rats.

The severity of possible adverse reactions to the combined immunotherapy and its therapeutic efficacy in a subject may be evaluated by the following tests: hematological tests, urinalysis and fecal test, and imaging examinations including CT scan. The efficacy of rhIL-12 can be estimated by measuring changes in tumor size in the subject. These tests can be done, as will be appreciated by the skilled artisan, at any time and in any combination during the course of the treatment.

Further, before administration of fusion cells to the subject, the fusion cells are washed to reduce contaminations with cytokines, such as GM-CSF, IL-4, and TNF-alpha. In a specific embodiment of the invention, contamination with cytokines does not amount to more than 10⁻⁹ gram cytokine per 10⁶ fusion cells. In a preferred embodiment, the fusion cells to be administered to a subject are suspended in about 1 mL of physiological saline, and injected subcutaneously to the subject. As the injection site, the groin area is chosen as it is rich in lymph nodes.

5.9 Administration Schedule

In a specific embodiment, fusion cells and rhIL-12 are administered in cycles. Each cycle consists of two weeks. In the first week of a cycle, 1-5×10⁵ fusion cells are administered first, followed by the administration of 30 ng/kg rhIL-12, followed by another administration of 30 ng/kg rhIL-12. In a specific embodiment rhIL-12 is administered on days 3 and 7 of the week 1 of the cycle. In the second week of the cycle, neither fusion cells nor rhIL-12 is administered. In a preferred embodiment, the treatment of a subject consists of three cycles. In another preferred embodiment, the treatment of a subject consists of four or five cycles. In a specific embodiment the cycle is repeated six times. The number of cycles depends on the condition of the patient, and can be determined by the skilled artisan according to the individual circumstances.

Throughout the course of the combined immunotherapy the subject is monitored closely. Several test are conducted to evaluate the state of the subject receiving the treatment. Such tests include, but are not limited to, hematological tests, urinalysis and fecal test, and imaging examinations including CT scan. Further, the efficacy of the treatment can be tested by monitoring the size of the tumor of the subject receiving the combined immunotherapy treatment.

Throughout the course of the combined immunotherapy the subject receiving the treatment is monitored for possible side effects. Side effects caused be administering rhIL-12 have been discussed in Section 5.7.

In a specific embodiment, fusion cells are kept in cell culture for up to 10 days prior to administration to the patient in need thereof. In a specific embodiment, fusion cells are kept in X-VIVO-15 medium (BioWhittaker, Walkersville, MD) supplemented with 10 ng/ml GM-CSF (Becton Dickinson), 30 U/ml IL-4 (Becton Dickinson), and 20 ng/ml TNF-α (Becton Dickinson).

In another specific embodiment, fusion cells are kept in RPMI medium (Sigma, St. Louis, Mo.) supplemented with 10 ng/ml GM-CSF (Becton Dickinson), 30 U/ml IL-4 (Becton Dickinson), and 20 ng/ml TNF-α (Becton Dickinson). In this embodiment, fusion cells do not have to be generated before each injection but can be obtained from the culture.

5.10 Effective Dose

The compositions can be administered to a subject at therapeutically effective doses to treat or prevent cancer. A therapeutically effective amount refers to that amount of the fusion cells sufficient to ameliorate the symptoms of such a disease or disorder, such as, e.g., cause or commence regression of a tumor. Effective doses (immunizing amounts) of the compositions of the invention may also be extrapolated from dose-response curves derived from animal model test systems. The precise dose of fusion cells to be employed in the pharmaceutical formulation will also depend on the particular type of disorder being treated. For example, if a tumor is being treated, the aggressiveness of the tumor is an important consideration when considering dosage. Other important considerations are the route of administration, and the nature of the patient. Thus the precise dosage should be decided according to the judgment of the practitioner and each patient's circumstances, e.g., the immune status of the patient, according to standard clinical techniques.

In a specific embodiment, for example, to treat a human tumor, a fusion cell-cytokine composition formed by cells of the tumor fused to autologous DCs at a site away from the tumor, and preferably near the lymph tissue. The administration of the composition may be repeated after an appropriate interval, e.g., every 3-6 months, using approximately 1×10⁸ cells per administration.

The present invention thus provides a method of immunizing a mammal, or treating or preventing cancer in a mammal, comprising administering to the mammal a therapeutically effective amount of a fusion cell-cytokine composition of the present invention.

In a preferred embodiment of the invention, rhIL-12 is administered at a dose between about 10 ng/kg to about 100 ng/kg body weight of the subject to whom the substance is to be administered. In another preferred embodiment, the dose of administration of rhIL-12 is 30 ng/kg body weight.

In a preferred embodiment, 1-5×10⁵ fusion cells are administered per injection. Before injection of the fusion cell, the safety of the fusion cells is monitored and confirmed. In a specific embodiment, the pH of the medium in which the fusion cells are cultured is measured. The pH should be between 7.0 and 7.4. In specific embodiments, the morphology of the fusion cells is analyzed by microscopy.

### 6. EXAMPLES

6.1 Vaccination with Dendritic Cells and Glioma Cells Against Brain Tumors

In the present example, the therapeutic use of dendritic cells fused to glioma cells against tumors in the brain, an immunologically privileged site, was investigated. Prior immunization with fusion cells (FCs) resulted in prevention of tumor formation upon challenge with glioma cells in the flank or in the brain. Efficacy was reduced when studies were performed in mice depleted of CD8⁺ cells. In a treatment model, FCs were injected subcutaneously after tumor development in the brain. Administration of FCs alone had limited effects on survival of brain tumor-bearing mice. Importantly, however, administration of FCs and recombinant IL-12 (rIL-12) remarkably prolonged survival of mice with brain tumors. CTL activity against glioma cells from immunized mice was also stimulated by co-administration of FCs and rIL-12 compared with that obtained with FCs or rIL-12 alone. These data support the therapeutic efficacy of combining fusion cell-based vaccine therapy and rIL-12.

6.1.1 Materials and Methods

Cell Lines Agents and Animals

The mouse glioma cell line, SR-B10.A, was maintained as monolayer cultures in DMEM (Cosmo Bio, Tokyo, Japan) supplemented with 100 U/ml penicillin, 0.1 mg/ml streptomycin, and 10% heat-inactivated fetal bovine serum (FBS; GIBCO, Gaithersburg, Md.). Yac-1 cells, obtained from RIKEN CELL BANK (Tsukuba, Japan), were maintained in RPMII640 (Cosmo Bio) with 10% FBS.

Recombinant mouse IL-12 (rmIL-12) was kindly provided by Genetics Institute, Cambridge, Mass.

Female B10.A mice, purchased from Sankyo Laboratory Inc. (Shizuoka, Japan), were maintained in a specific pathogen-free room at 2513° C. Mice were used at 8 weeks of age.

Fusions of Dendritic and Tumor Cells

Bone marrow was flushed from long bones of B10.A mice, and red cells were lysed with ammonium chloride (Sigma, St. Louis, Mo.). Lymphocytes, granulocytes and DCs were depleted from the bone marrow cells and the cells were plated in 24-well culture plates (1×10⁶ cells/well) in RPMI 1640 medium supplemented with 5% heat-inactivated FBS, 50 μM 2-mercaptoethanol, 2 mM glutamate, 100 U/ml penicillin, 100 pg/ml streptomycin, 10 ng/ml recombinant murine granulocyte-macrophage colony stimulating factor (GM-CSF; Becton Dickinson, San Jose, Calif.) and 30 U/ml recombinant mouse interleukin-4 (IL-4; Becton Dickinson). On day 5 of culture, nonadherent and loosely adherent cells were collected and replated on 100-mm petri dishes (1×10⁶ cells/ml; 10 ml/dish). GM-CSF and IL-4 in RPMI medium were added to the cells and 1×10⁶ DCs were mixed with 3×10⁶ irradiated (50 Gy, Hitachi MBR-1520R, dose rate: 1.1 Gy/min.) SR-B10.A cells. After 48 h, fusion was started by adding dropwise for 60 sec, 500 μl of a 50% solution of polyethylene glycol (PEG; Sigma). The fusion was stopped by stepwise addition of serum-free RPMI medium. FCs were plated in 100-mm petri dishes in the presence of GM-CSF and IL-4 in RPMI medium for 48 h.

Flow Cytometry

Tumor cells (3×10⁶) were harvested and washed twice with phosphate-buffered saline (PBS; Cosmo Bio). PKH26 (2 μl; Sigma) was added to the tumor cells and the mixture was kept at room temperature for 5 mm. Then, 500 μl FBS was added to stop the reaction. Cells were washed twice using PBS and resuspended in 500 μl of PBS. Single cell suspensions of DCs and FCs were prepared, washed, resuspended in buffer (1% BSA, 0.1% Sodium azide in PBS) and stained with an FITC-labeled anti-mouse CD80 monoclonal antibody (Pharmingen, San Diego, Calif.) for 30 mm at 4° C. Stained cells were analyzed using a FACScan flow cytometer (Becton Dickinson, San Jose, Calif.).

Animal Models

FCs were washed twice with PBS, then suspended in PBS at a density of 1×10⁶ ml. FCs (3×10⁵) were subcutaneously (s.c.) inoculated into the flank of B10.A mice on days 0 and 7. Subsequently, tumor cells (1×10⁶) were inoculated s.c. into the opposite flank on day 14. In the brain tumor model, 1×10⁴ SR-B10.A. Tumor cells were stereotactically inoculated into the right frontal lobes of the brains of syngeneic mice on day 14 after immunization with FCs.

In the treatment model, 1×10⁴ tumor cells were stereotactically inoculated into the brains (day 0) followed by s.c. injection of FCs (3×10⁵) on days 5 and 12. In certain experiments, rmIL-12 was injected intraperitoneally (i.p.). Autopsy was performed on deceased mice.

Assay of Cytolytic Activity

The cytolytic activity of activated spleen cells (SPC) was tested in vitro in a ⁵¹Cr release assay. Single cell suspensions of SPC from individual mice were washed and resuspended in 10% FCS-RPMI at a density of 1×10⁷/ml in six-well plates (Falcon Labware, Lincoln Park, N.J.) (Day 0). After removing adherent cells, 10 U/ml of recombinant human IL-2 was added to the cultures every other day. Four days after culture initiation, cells were harvested and cytotoxic T cells (CTL) activity was determined. Target cells were labeled by incubation with ⁵¹Cr for 90 mm at 37° C., then co-cultured with effector lymphocytes for 4 hours. The effector:target ratio ranged from 10:1 to 80:1. All determinations were made in triplicate and percentage lysis was calculated using the formula: (experimental cpm−spontaneous cpm/maximum cpm−spontaneous cpm)×100%.

Antibody Ablation Studies

In vivo ablation of T-cell subsets was accomplished as previously described (Kikuchi et al., 1999, Int J Cancer, 80:425-430). Briefly, 3×10⁵ FCs were inoculated subcutaneously into the flank of B10.A mice on days 0 and 7. Subsequently, tumor cells (1×10⁶) were inoculated into the opposite flank on day 14. The rat monoclonal antibodies anti-mCD4 (ATCC hybridoma GK1.5), anti-mCD8 (ATCC hybridoma 56.6.73), anti-asialo GMI (Wako Pure Chemicals, Tokyo, Japan) or normal rat IgG was injected i.p. (0.5 mg/injection/mouse) on days 7, 10, 14 and 17.

Immunofluorescence Staining

Tumor cells (1×10⁴) were stereotactically inoculated into the brains (day 0) followed by subcutaneous (s.c.) injection of FCs (3×10⁵) or irradiated glioma cells (3×10⁵) on day 3 as a control. After sacrificing the mouse on day 17, we fixed the brain in fixation buffer (1% paraformaldehyde and 0.1% glutaraldehyde in PBS) for 10 mm. Sections (6 μm thickness) were incubated overnight at 4° C. with the first antibody, anti-glial fibrillary acidic protein (anti-GFAP; Zymed Laboratories, San Francisco, Calif.). The primary antibody was detected by FITC-conjugated goat anti-rabbit IgG (Jackson ImmunoResearch Laboratories, West Grove, Pa.) in a 2 h incubation at room temperature. Subsequently, sections were incubated overnight at 4° C. with anti-CD4-PE (Pharmingen) or anti-CD8-PE (Pharmingen) antibody.

Data Analysis

Calculated tumor sizes were compared using a two-sample t test. Survival was evaluated by generation of Kaplan-Meier cumulative hazard plots and Wilcoxon analysis. Differences were considered significant at p<0.05.

6.1.2 Results

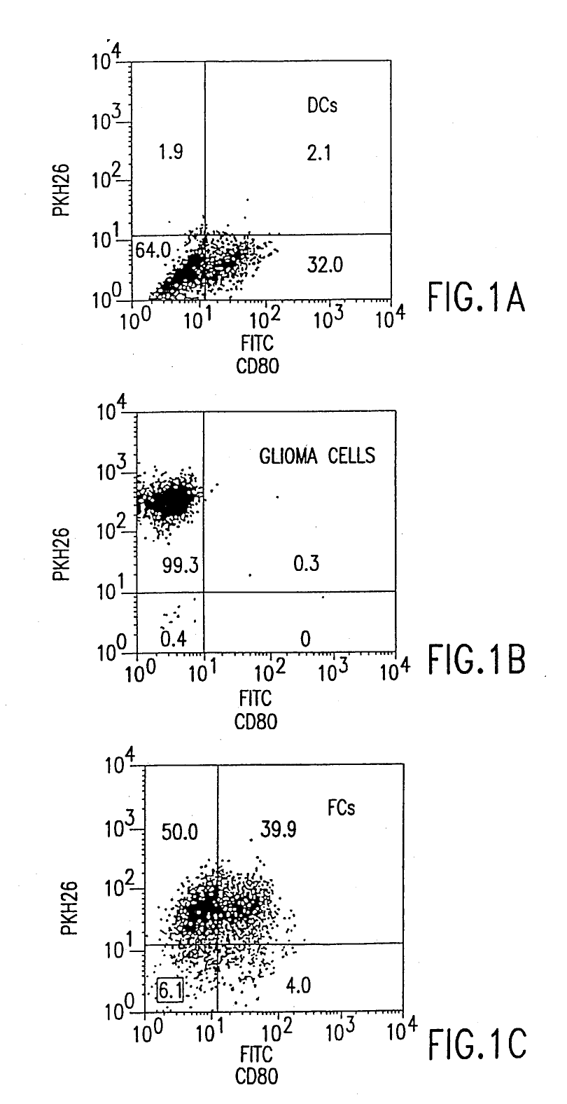

DCs and glioma cells were fused after incorporation of PKH26 into glioma cells. DCs were stained by FITC-labeled anti-CD80 monoclonal antibody. FIG. 1A shows that 34% of DCs were stained by anti-CD80 monoclonal antibody. More than 95% of glioma cells were positive for PKH26 (FIG. 1B). The percentage of double positive cells (39.9%; FIG. 1C) was nearly identical to the percent of CD80-positive DCs and 10% of FCs were PKH26-negative, suggesting that most DCs were fused with glioma cells.

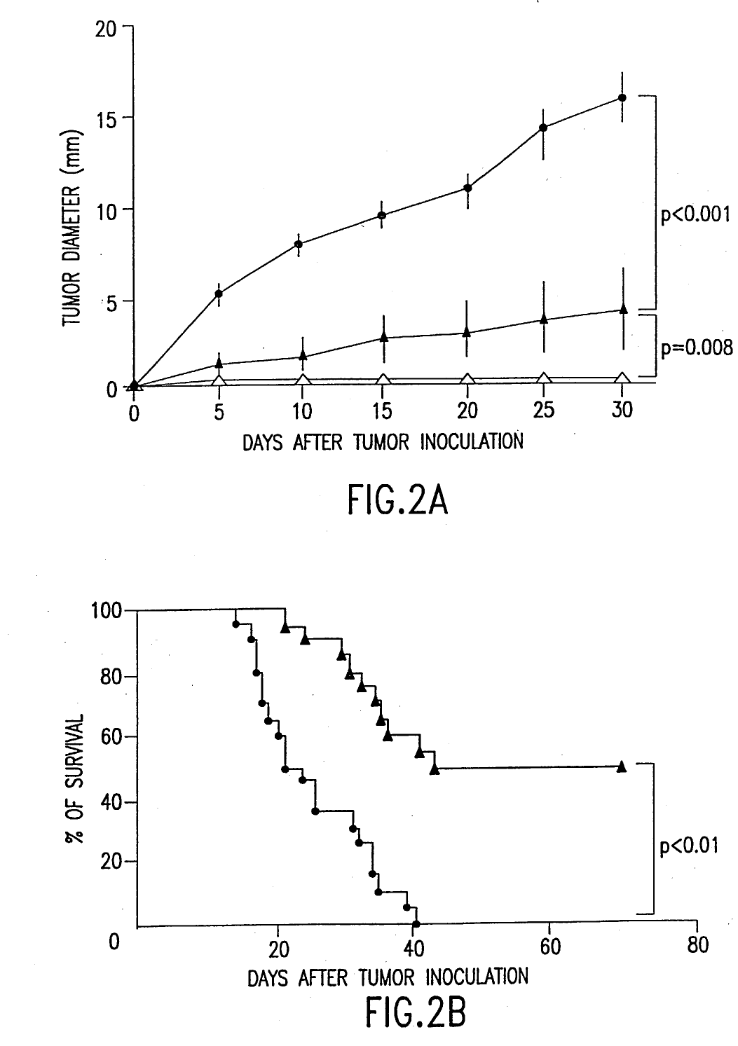

The antitumor effects of prior immunization with FCs on subcutaneous gliomas was examined. FCs, DCs, or irradiated parental cells as a control (1×10⁶) were injected s.c. into syngeneic mice on days 0 and 7 (n=11 in each group). On day 14, 1×10⁶ parental cells were inoculated s.c. into the opposite flank. Within two weeks, the inoculated tumor cells caused large tumors in all mice injected with irradiated parental cells. All of the mice died within six weeks. In contrast, none of the mice immunized with FCs died within six weeks. Whereas six of 11 mice immunized with DCs developed tumors, none of 11 mice immunized with FCs developed a palpable tumor (FIG. 2A).

We also investigated the antitumor effects of prior immunization with FCs on gliomas in the brain. After immunization with FCs on days 0 and 7, 1×10⁴ tumor cells were stereotactically inoculated into the right frontal lobe of the brain (day 14). These mice were observed for 70 days. Half of the mice immunized with FCs survived longer than 70 days (n=20 in each group; p<0.01) (FIG. 2B). All control mice died within 6 weeks. Autopsy was performed on all mice. Large tumors had developed in the dead mice, but not in the surviving mice. These findings indicate that immunization with FCs prevents the development of glioma cell tumor in the flank and in the brain.

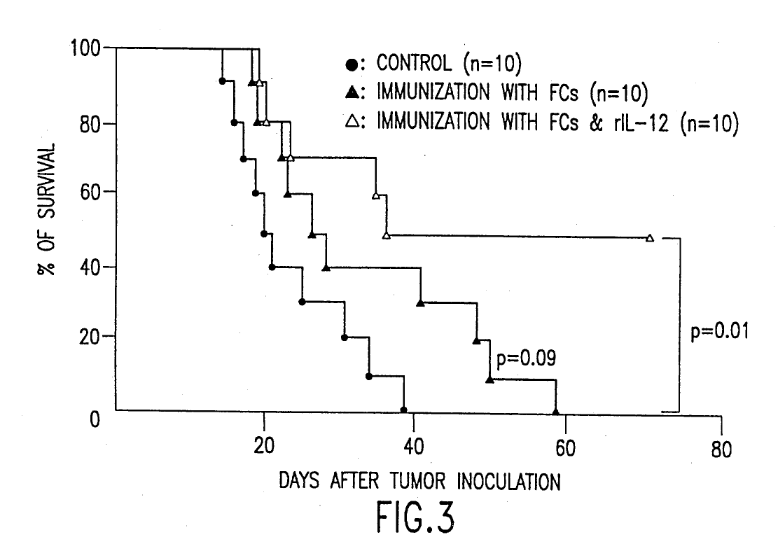

As an experimental treatment model, FCs were injected after brain tumor development. Tumor cells (1×10⁴) were stereotactically inoculated into the right frontal lobes of the brains of syngeneic mice (day 0). On days 5 and 12, 3×10⁵ FCs were inoculated s.c. Although, vaccination with FCs prolonged the survival of tumor-bearing mice (n=15 each; FIG. 3), the difference was not significant (p>0.05). Inoculation of DCs alone had no effect on survival (data not shown). We then analyzed antitumor effects of combined FCs and rmIL-12 therapy. Tumor cells (1×10⁴) were stereotactically inoculated into the brains of syngeneic mice (day 0). On days 5 and 12, 3×10⁵ FCs were inoculated s.c. All mice were given an i.p. injection of 0.5 μg/100 μl rmIL-12 or 100 μl saline every other day for two weeks (3.5 μg/mouse total) starting on day 5. Vaccination with both FCs and rIL-12 prolonged survival in comparison with the control (p=0.01; FIG. 3). Five of ten mice treated with FCs and rIL-12 survived over 70 days. The difference in survival rates between the controls and mice treated with rmIL-12 alone or both DCs and rmIL-12 was not statistically significant (data not shown). These results demonstrate that rmIL-12 potentiates the antitumor effects of the FC composition.

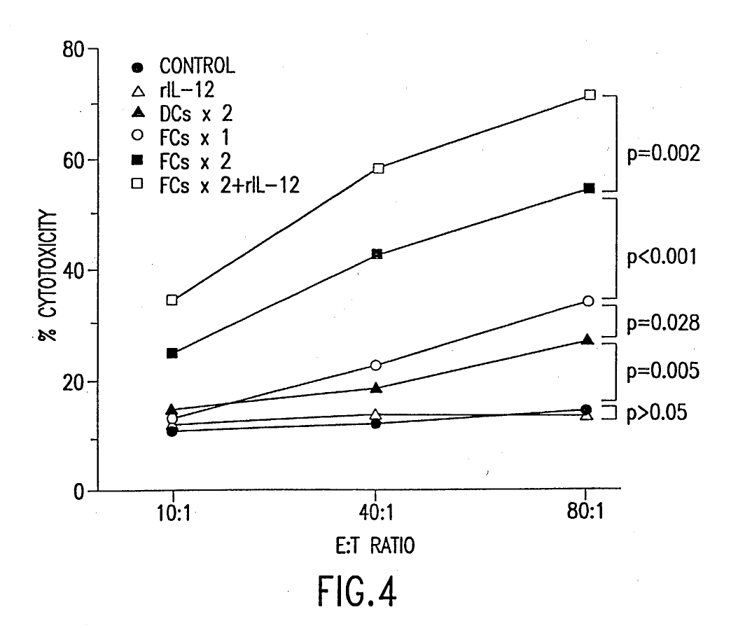

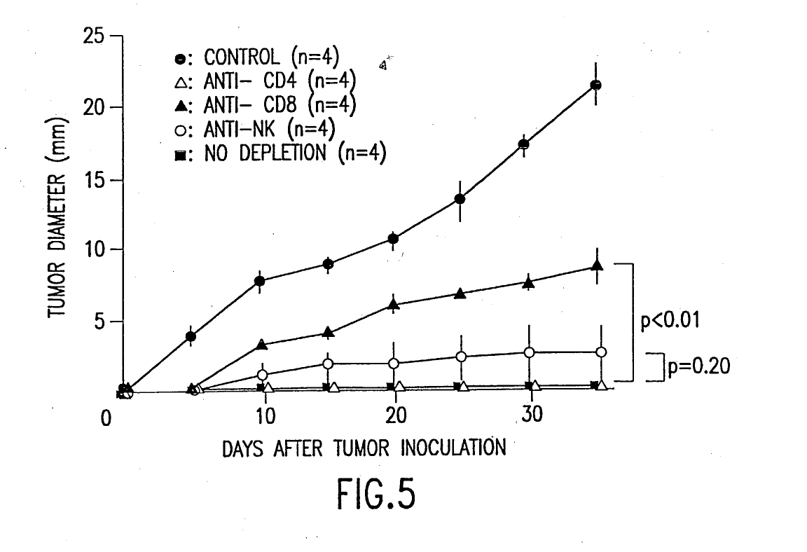

CTL activity was analyzed by a ⁵¹Cr release assay. After immunization with FCs (on day 0 and/or 7) and/or rIL-12 (every other day for 10 days starting on day 7; 2.5 pg/mouse total), splenocytes (SPCs) were separated from untreated mice and the mice immunized with FCs once or twice. FIG. 4 shows that CTL activity on tumor cells from immunized mice, especially mice injected with rIL-12 and immunized with FCs twice, was considerably increased compared with the control and others and that antitumor activity on Yac-1 cells from treated mice did not significantly increase (data not shown). These results suggest that vaccination with FCs induced antitumor activity and that the cytolytic activity of SPCs from treated mice was tumor-specific.

In addition, lymphocyte subsets were depleted by administering anti-CD4, anti-CD8, anti-asialo GMI, or control rat IgG into mice given injections of glioma cells and FCs. On days 0 and 7, FCs were subcutaneously inoculated into the flank. Subsequently, on day 14 parental cells were inoculated into the opposite flank. The mAbs were injected i.p. on days 7, 10, 14, and 17. The antitumor effect was reduced in mice depleted of CD8⁺ T cells (n=4 in each group; FIG. 5). The protection conferred by FCs was not abolished by CD4⁺ T or NK cell depletion. These results demonstrate that CD8⁺ T cells are required for the antitumor effect of FCs in this model.

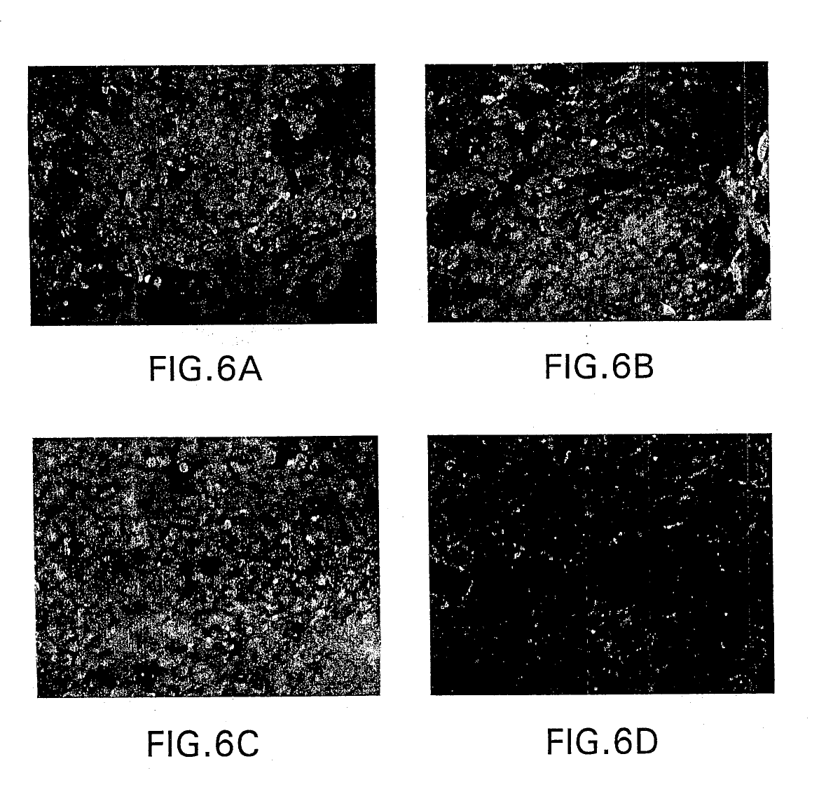

In the experimental treatment model, we analyzed whether CD4⁺ and/or CD8⁺ T cells were infiltrating into the brain tumor. Immunofluorescence analysis of the brain tumors showed that a few CD4⁺ and CD8⁺ T cells were present in the tumors of non-vaccinated mice (FIGS. 6A, B). In contrast, numerous CD4⁺ and CD8⁺ T cells were detectable in the tumors of vaccinated mice (FIGS. 6C, D). As reported previously, SRB10.A cells were positive for GFAP (10).

6.1.3 Discussion

Genetically engineered glioma cells can be used as APCs for vaccination against gliomas, but the antitumor effect is not sufficient to eradicate established brain tumors in the mouse model (Aoki et al., 1992, Proc Natl Acad Sci USA, 89:3850-4); Wakimoto, H. et al., 1996, Cancer Res, 56:1828-33). Therefore, a DC-based composition is a potential approach that could be used for the treatment of brain tumors. DCs lose the ability to take up antigens. Therefore, use of DCs requires efficient methods to incorporate TAAs into DCs. So far, several methods using DCs for the induction of antitumor immunity have been investigated: DCs pulsed with proteins or peptides extracted from tumor cells (Zitvogel et al., 1996; Nair et al., 1997, Int J Cancer, 70:706-15; Tjandrawan et al., 1998, J Immunother, 21:149-57), QCs transfected with genes encoding TAAs (Tuting et al., 1998, J Immunol, 160:1139-47), DCs cultured with tumor cells (Celluzi and Falo, 1998) and DCs fused with tumor cells (Gong et al., 1997, Nat Med, 3:558-61; Gong et al., 1998, Proc Natl Acad Sci USA, 95:6279-83; Lespagnard et al., 1998, Int J Cancer, 76:250-8; Wang et al., 1998, J Immunol, 161:5516-24). Since, 1) FCs can be used to induce antitumor immunity against unknown TAAs, 2) the common TAAs of gliomas have not been identified and 3) antitumor effects of FCs provide a more thorough cure than mixture of DCs and tumor cells, FCs may have an advantage as a potential therapeutic approach for malignant gliomas.

Although the effects of FCs on tumor cells in a mouse subcutaneous tumor model were previously reported (Gong et al., 1997, Nat Med, 3:558-61), the effects in the brain remained unclear. In our brain tumor model, systemic vaccination with FCs rendered tumor cells susceptible to rejection, which resulted in the establishment of systemic immunity and prolonged survival. The central nervous system (CNS) is generally considered an immunologically privileged site due to the lack of lymphatic drainage and the nature of the blood brain barrier in which tight junctions between cerebral vascular endothelial cells form a physical barrier to the passage of cells and antibodies (Cserr, H. F. and Knopf, P. M., 1992, Immunol Today, 13:507-12). However, the present study shows that systemic vaccination with FCs can be used to treat established brain tumors. Therefore, the brain may not be completely immuno-privileged or, alternatively, barriers to the immune system can be surmounted for certain tumors, resulting in crosstalk between systemic and focal immunity.

In the present study, vaccination with FCs alone prolonged survival of mice with brain tumors. We therefore reasoned that the immunization treatment schedule and method might be improved by injecting FCs with stimulatory cytokines. Indeed, administration of rmIL-12 enhanced the antitumor effect of FCs against mouse gliomas. IL-12, originally called natural killer cell stimulatory factor or cytotoxic lymphocyte maturation factor, enhances the lytic activity of NK/lymphokine-activated killer (LAK) cells, facilitates specific cytotoxic T lymphocyte (CTL) responses, acts as a growth factor for activated T and NK cells, induces production of IFN-γ from T and NK cells, and acts as an angiogenesis inhibitor (Brunda, M. J., 1994, J. Leukoc Biol, 55:280-8). Although IL-12 has the potential to be used as an immunomodulator in the therapy of malignancies and has been shown to significantly retard the growth of certain murine tumors (Gately et al., 1994, Int Immunol, 6:157-67); Nastala et al., 1994, J Immunol, 153:1697-706), systemic administration of rmIL-12 did not prolong the survival of mice with brain tumors (Kikuchi et al., 1999, Int J Cancer, 80:425-430), indicating that the antitumor effect of combined FCs and rmIL-12 therapy may be synergistic. There were few lymphocytes present in the brain tumors from control mice. Importantly, however, immunization with FCs substantially increased lymphocyte infiltration. In addition, at the tumor site, the concentration of tumor-derived immuno-suppressive factors (e.g. TGF-β, IL-10, prostaglandin E2) may be high, indicating that more potent CTL may be needed to cure brain tumors.

DCs can sensitize CD4⁺ T cells to specific antigens in a MHC-restricted manner. CD4⁺ T cells are critical in priming both cytotoxic T lymphocytes and antigen non-specific effector immune responses, implying that both CD4⁺ and CD8⁺ T cells are equally important in antitumor immunity. As reported previously, antitumor effects of cells fused with DCs and MC38 were mediated via both CD4⁺ and CD8⁺ T cells (Gong et al., 1997, Nat Med, 3:558-61). However, our results demonstrated that CD8⁺ T cells were required for the antitumor effect of FCs and that the role of CD4⁺ T cells less obvious. Okada et al. (1998, Int J Cancer, 78:196-201) reported that only CD8⁺ T cells were required for antitumor effects of peptide-pulsed DCs in a brain tumor model (Okada et al., 1998, Int J Cancer, 78: 196-201). Therefore, the cell type mediating the anti-tumor effects of DCs may not be universal, but rather dependent upon the experimental model. Histopathological findings showed that both CD4⁺ and CD8⁺ T cells were present in the brain tumors. It may be speculated that CTLs were already primed before starting the vaccination with FCs. That is, CD4⁺ T cells have already finished priming CTLs before immunization with FCs and pre-CTLs (primed CTLs) were stimulated by FCs, resulting in induction of activated CTLs and acquisition of antitumor activity.

In conclusion, our data suggest that vaccination with FCs and rIL-12 can be used to treat malignant gliomas in a mouse model. In the present study, we fused DCs with an established tumor cell line. However, for clinical application, DCs should be fused with removed tumor materials or primary cultured cells. Future research will focus on characterizing the antitumor activities of cells fused with DCs and primary cultured human glioma cells.

6.2 Treatment with Tumor Cell-Dendritic Cell Hybrids in Combination with Interleukin-12

Hepatocellular carcinoma (HCC) is one of the most common cancers in the world, especially in Asian and African countries. While this disease is rare elsewhere (a), recent reports have indicated that HCC is now increasing in Western countries (El-Selag et al., 1999, N. Engl. J. Med., 340:745-750). Epidemiological and prospective studies have demonstrated a strong etiological association between hepatitis B virus (HBV) and/or hepatitis C virus (HCV) infection and HCC (Ikeda et al., 1993, Hepatology, 18:47-5; Obata et al., 1980, Int. J. Cancer, 25:741-747; Saito et al., 1990, Proc. Natl. Acad. Sci. USA, 87:6547-6549). In Japan, about 76% of HCC patients had chronic HCV infection and 78% of them had liver cirrhosis (Liver Cancer Study Group of Japan, 1998). The reduction in functional reserve due to the coexisting liver cirrhosis has limited surgical resection of the tumor. Consequently, treatment has involved cancer chemotherapy, transcatheter arterial embolization, transcatheter arterial chemotherapy, percutaneous ethanol injection and percutaneous microwave coagulation therapy. However, the recurrence rate after these therapies is high (Liver Cancer Study Group of Japan, 1998; Tarao et al., Cancer, 79:688-694), probably because of the insufficient therapeutic effect and multicentric development of HCC in a cirrhotic liver.

In the present study, we show that the growth of HCC tumors is prevented by vaccination of DCs fused to HCC cells prior to inoculation of HCC cells. In addition, treatment of established HCC tumors with DC/HCC requires co-administration with IL-12. Importantly, IL-12 can also enhance the effectiveness of fusion cell-based immunotherapy.

6.2.1 Materials And Methods

Mice, Tumor Cell Lines Cytokines and Antibodies

Female BALB/c mice, 8 to 10 weeks old, were purchased from Nippon SLO (Sbizuoka, Japan). A murine HCC cell line, BNL, was kindly provided by Dr. S. Kuriyama (Nara Medical University, Nan., Japan). C26, a colon carcinoma cell line of BALB/c mouse, was provided from Tyugai Pharmaceutical Company, Tokyo. Murine recombinant IL-12 (mrIL-12) was kindly provided by Genetics Institute, Cambridge, Mass. Human recombinant IL-2 (hrIL-2) was kindly provided by Sbionogi Pharmaceutical Company, Tokyo. Rat monoclonal antibodies against murine CD4, CD8, H-2K_{d} and I-A_{d}/I-E_{d} were purchased from Pharmingen, San Diego.

Preparation of DCs

DCs were prepared with the method described by Inaba et al (Inaba et al., 1992, J. Exp. Med., 176:1693-1702) with modifications. Briefly, bone marrow cells were obtained from the femur and tibiae of female BALB/c mice (8 to 10 weeks old). Red blood cells were lysed by treatment With 0.83% ammonium chloride solution. The cells were incubated for 1 hour at 3700 on a plate coated with human γ-globulin (Cappel, Aurora, Ohio) (Yamaguchi et al., 1997, Stem Cell, 15:144-153). Nonadherent cells were harvested and cultured on 24-well plates (10⁵ cells/ml/well) in medium containing 10 ng/ml murine recombinant granulocyte/macrophage) colony-stimulating factor (GM-CSP) (Becton-Dickinson, Bedford, Mass.) and 60 U/mm of recombinant murine IL-4 (Becton-Dickinson). After 5 days of culture, nonadherent or loosely attached calls were collected by gentle pipetting and transferred to a 100-nun Petri dish. Floating cells, which included many DCs, were collected after overnight culture. The cells obtained in this manner exhibited dendritic features and cell surface expression of MHC class 1, class II CD80, CD86, CD54 but not CD4, CD8 and CD4SR.

Fusion of DCs and BNL Cells

Fusion of DCs and BNL cells were performed according to Gong et al. (Gong et al., 1997, Nat. Med., 3:558-561) with modifications. Briefly, BNL cells were irradiated in the 35 Gy, mixed with DCs at a ratio of 1:3 (BNL:DC) and then centrifuged. Cell pellets were treated with 50% polyethylene glycol (PEG 1460, Sigma Chemical Co., St. Louis, Mo.) for 1 minute at 37° C., after which the PEG solution was diluted with warm RPMI 1640 medium. The PEG treated cells were cultured overnight at 37° C. in medium containing GMCSF and IL-4.

FACS Analysis of the Cells

To determine the efficiency of cell fusion, BNL cells were stained with PKH-26 (red fluorescence) and DCs were stained with PKH-2GL (green fluorescence). The cells stained with the fluorescent dyes were treated with PEG and cultured overnight as described above. The fusions were also stained with phycoerythin (PE) or fluorescein isothiocyanate (FITC) conjugated with monoclonal antibodies against I-A^{d}/I-E^{d}, CD80, CD86 and CD54 (Pharmingen, San Diego). Fluorescence profiles were generated with a FACSCalibur flow cytometer (Becton-Dickinson, San Jose, Calif.). Histograms and density plots were generated with the Cell Quest software package (Becton Dickinson, San Jose, Calif.).

Scanning Electron Microscopy

Cells were fixed with 1.2% glutaraldehyde in 0.1 M phosphate buffer (pH 7.4). Fixed cells were attached to slides previously coated with 0.1% poly-L-lysine, dehydrated in ascending concentrations of ethanol, treated with isoamyl acetate and critical-point dried with liquid CO₂. Specimens were coated with vacuum-evaporated, iron-sputtered gold and observed with a JSM-35 scanning electron microscope (Japan Electric Optical Laboratory, Tokyo, Japan) at an accelerating voltage of 10 kV.

Injection of the Fusions to Mice and Administration of IL-12

In tumor prevention studies, DC/BNL fusions were suspended in phosphate-buffered saline (PBS) and injected into the tail vein of mice (4×10⁵ cells/mouse), twice, at an interval of 2 weeks. One week after the second immunization, tumor challenge was performed by subcutaneous injection of 10⁶ BNL cells. The mice were monitored each week for the development of tumor by measurement of tumor size (≧3 mm scored as positive). The control mice received phosphate-buffered saline (PBS), irradiated BNL cells (10⁵/mouse), DCs (3×10⁵/mouse) or mixture of irradiated BNL cells and DCs (4×10⁵/mouse, DC:BNL ratio 3:1) instead of the DC/BNL fusions, and were examined for development of the tumor as those which received the fusions. Each group consisted of 10 mice.

In treatment studies, the mice were divided into four groups. Ten mice in each group had BNL cells inoculated subcutaneously. In group A, DC/BNL fusions were injected subcutaneously on days 3 and 10 after inoculation of BNL cells. IL-12, dissolved in PBS containing 0.3% bovine serum albumin, was injected intraperitoneally on 2, 4 and 6 days after the first inoculation of the fusions and 3 and 5 days after the second inoculation. The mice in group B were treated in the same way as those in group A except that they did not receive IL-12. The mice in group C were treated in the same way as those in group A except that they did not receive the fusions. The mice in group D were treated in the same way as those in group A except that they received neither IL-12, nor the fusions.

Assay of Lytic Activity of Splenocytes Against BNL Cells

Splenocytes were obtained by gentle disruption of the spleen on a steel mesh and depletion of red blood cells by hypotonic treatment. Splenocytes from the mice were cultured in RPMI-1640 medium supplemented with 10% heat inactivated fetal calf serum (FCS) containing 50 U/ml of human recombinant IL-2 for 4 days. BNL cells (10⁴ cells/well) were labeled with ⁵¹Cr and incubated in RPMI-1640 medium supplemented with 10% heat inactivated FCS with splenocytes (effector cells) at the indicated effector target ratios in a volume of 200 ul in triplicate in a 96 multiwell plate for 4 hours at 37° C. After incubation, 100 μl of supernatant was collected and the percent specific ⁵¹Cr release was calculated with the following formula: percent ⁵¹Cr release=100×(cpm experimental cpm spontaneous release)\(cpm maximum release−cpm spontaneous release), where maximum release was that obtained from target cells incubated with 0.33N HCl and spontaneous release was that obtained from target cells incubated without the effector cells. For assessing inhibition of lytic activity by rat monoclonal antibodies against murine CD4, CD8, H-2K^{d}, I-A^{d}/I-E^{d}, 50 ug/ml of each antibody was added to the culture during the 4 hour incubation.

Immunohistochemical Studies

Immunofluorescent staining was performed by direct immunofluorescence. Frozen sections of tumor tissue were made and fixed with acetone for 10 minutes at room temperature. After washing with PBS, the sections were incubated in 10% normal goat serum in PBS for 20 minutes at room temperature, and then with the PE or FITC-labeled antibody in 10% normal goat serum in PBS for 2-3 hours at room temperature in a dark box. Sections were washed with PBS, mounted and observed under a fluorescent microscope.

6.2.2 Results

Characteristics of Fusions of DCs and BNL Cells

DCs and BNL cells were combined, treated with PEG and incubated overnight. Nonadherent and adherent cells obtained from PEG-treated cells exhibited dendritic features and epithelial characteristics, respectively, under a phase contrast microscope. Nonadherent cells expressed DC markers, I-A^{d} (MHC class II) and CD11c, by FACS analysis (data not shown). The finding that the adherent cells are negative for I-A^{d} and CD11c expression indicated that BNL cells were in the adherent cell fraction.

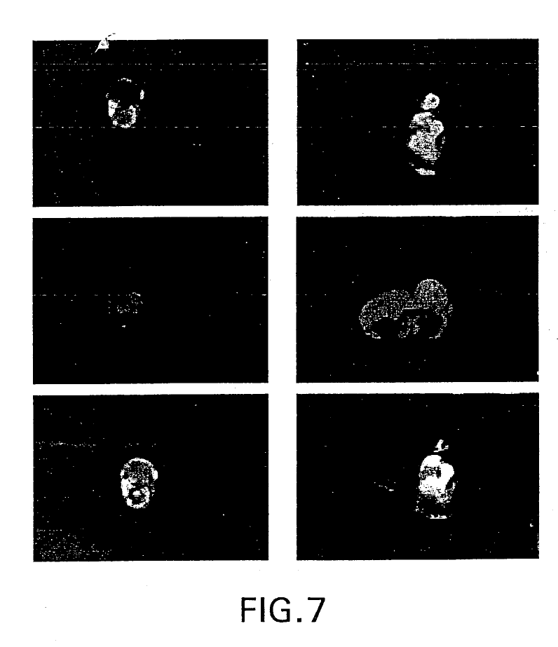

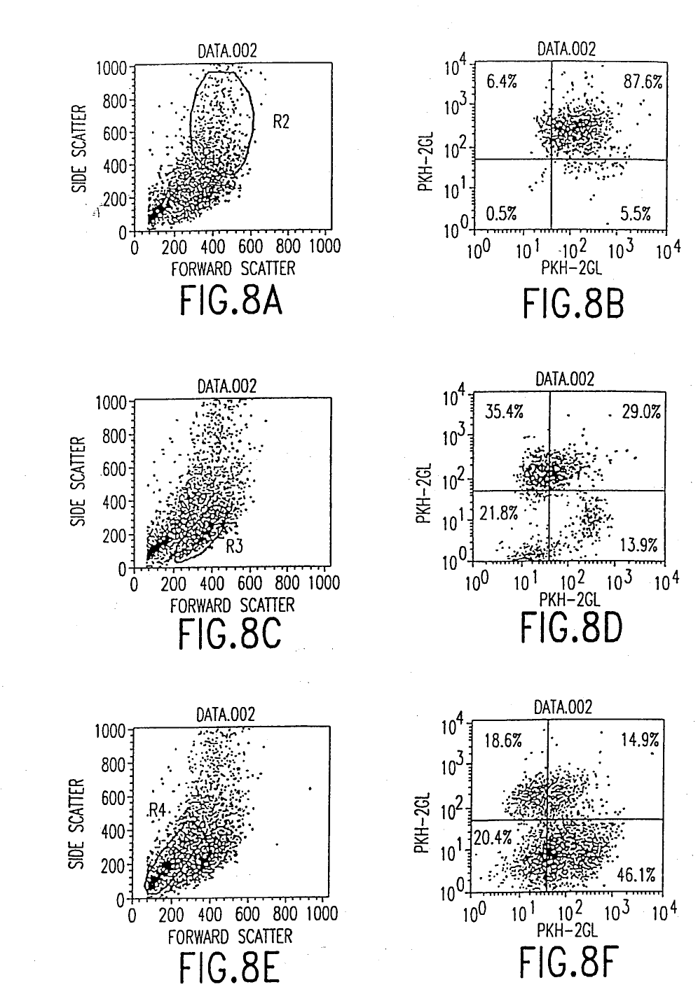

Prior to PEG treatment, DCs were treated with an FITC conjugated antibody against CD11c and BNL cells were stained with PKH-26. The cells were fused by PEG treatment and observed under a fluorescence microscope. Cells stained with both FITC (green) and PKH-26 (red) were observe among the PEG-treated cells (FIG. 7). For determination of the fusion efficacy, DCs and BNL cells were stained with fluorescent dyes, PKH-2GL and PKH-26, respectively, and then treated with PEG. By FACS analysis, cells stained with both PKH-2GL and PKH-26, which were considered to be fusions of DCs and BNL cells, are shown in upper area of cell scattergram with high forward scatter and high side scatter (FIG. 8). The cell fraction of high and moderate forward scatter and low side scatter contained many non-fused BNL cells, which those of low forward scatter and low side scatter contained non-fused DCs and non-fused BNL cells (FIG. 8). About 30% of the nonadherent cells were fusions as judged from the width of area of double positive cells occupying in the whole scattergram.

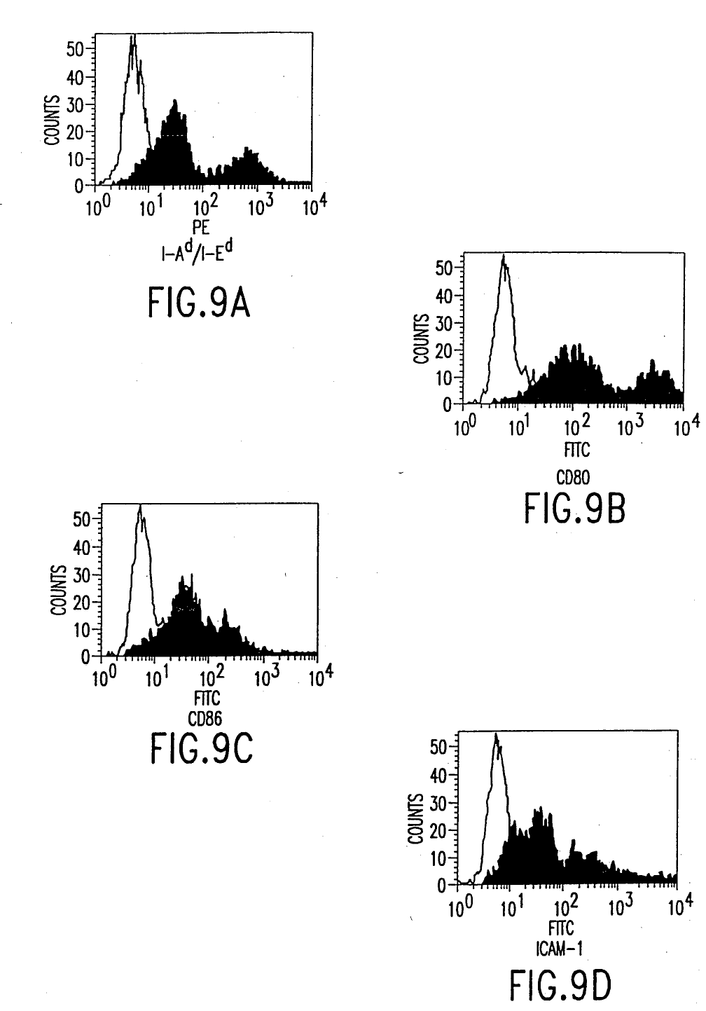

Phenotypes of the fusions were analyzed by FACS. The cell fraction positive for both PKH-2GL and PKH-26 were gated on scattergram and examined for antigen expression. I-A^{d}/I-E^{d} (MCH class II), CD80, CD86 and CD54 molecules, which are found on DCs, were expressed by the fusions (FIG. 9).

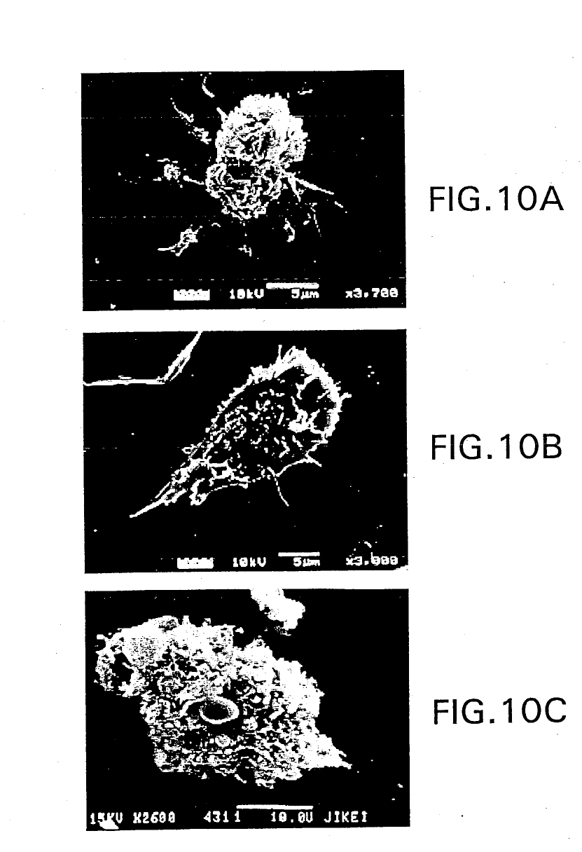

In addition, scanning electron microscopy showed that BNL cells express short processes on a plain cell surface, whereas DCs had many long dendritic processes. The nonadherent fusion cells were large and ovoid with short dendritic processes (FIG. 10).

Effect of Vaccination with DC/BNI, Fusions on Prevention of Tumor Development.

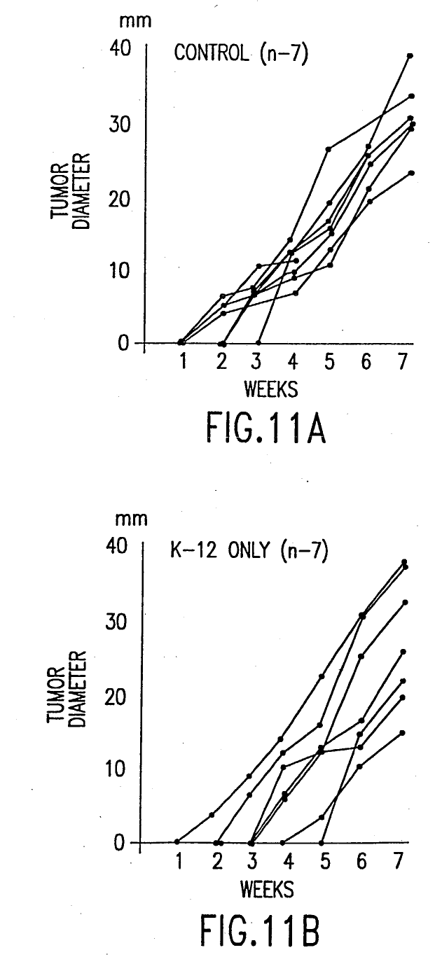

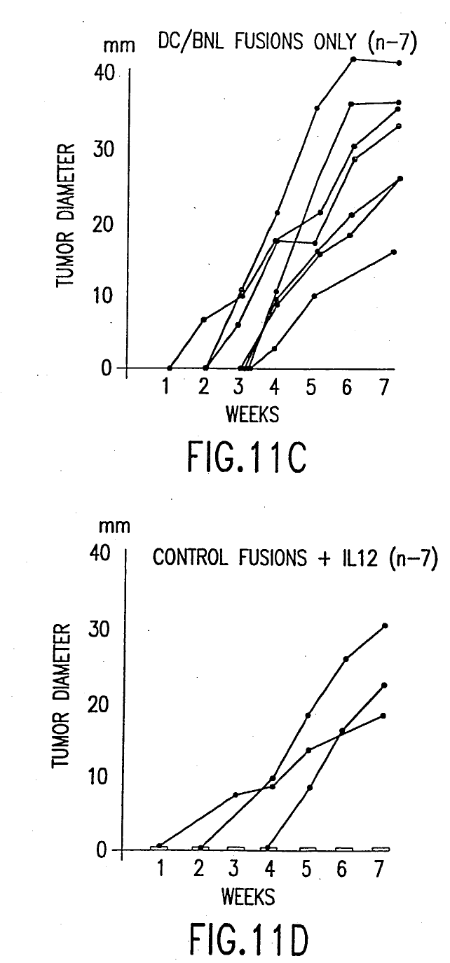

Vaccination with DC/BNL fusions resulted in the rejection of a challenge with BNL cells inoculated in BALB/c mice. By contrast, injection of only DCs or only irradiated BNL cells failed to prevent the development and growth of tumors (FIG. 11). Injection of mixture of DCs and BNL cells, in numbers corresponding to those used to produce the fusions, transiently inhibited tumor growth, but after 4 weeks, tumors grew at rates comparable to controls. The finding that C26 colon carcinoma cells were not rejected by prior injection of DC/BNL fusions indicated that the immunity induced by DC/BNL fusions was specific for BNL cells (data not shown).

Effects of Vaccination with DC/BNL Fusions on Treatment of Pre-Established BNL Tumors.

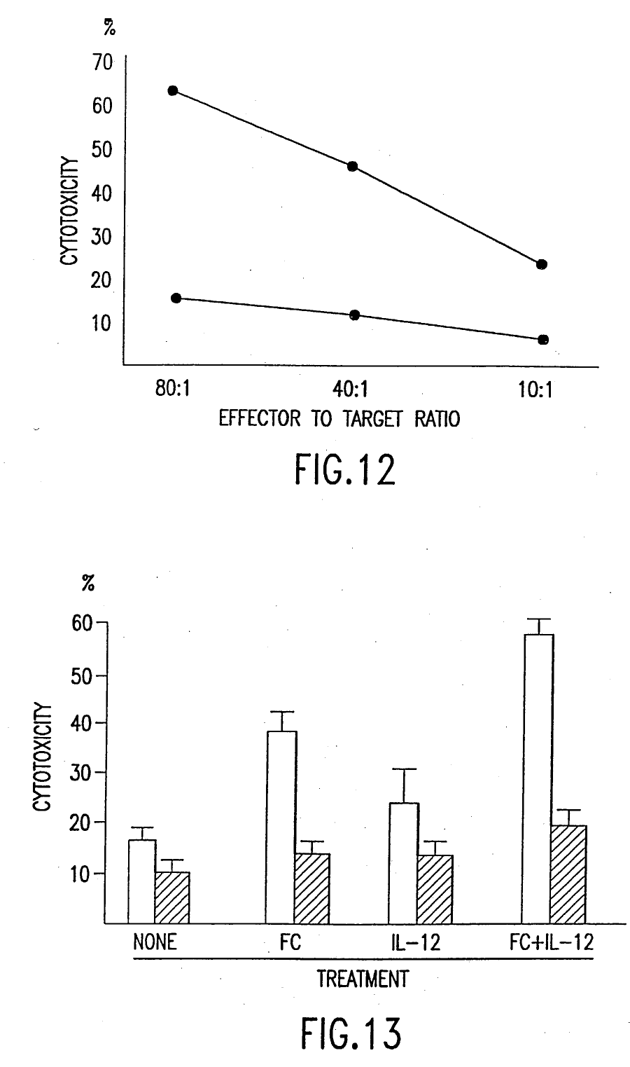

BNL cells (10⁶/mouse) were inoculated 3 days before treatment with DC/BNL fusions. The effect of treatment with DC/BNL fusion cells alone against BNL tumor was not significant (FIG. 12). In addition, systemic administration of IL-12 (200 ng/mouse, intraperitoneal) alone had no significant therapeutic effect against growth of BNL cells; tumors were observed in all mice within 7 weeks after inoculation. However, injection of DC/BNL fusions followed by administration of IL-12 elicited a significant antitumor effect. Four of the seven mice showed no BNL tumor development. Thus, tumor incidence 7 weeks after BNL cell inoculation was 43% (3/7). Neither increasing nor decreasing the dose of IL-12 in this protocol improved the antitumor effect.

Lytic Activity of Splenocytes Against BNL Cells in Mice Treated with DC/BNL Fusions and IL-12.

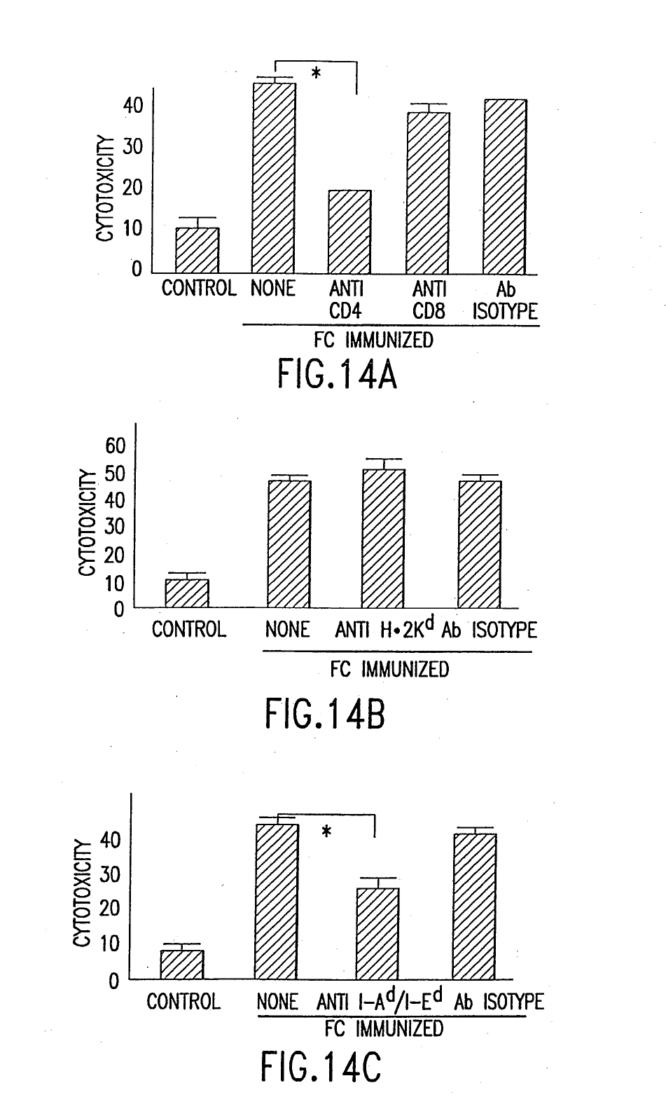

Significant cytolytic activity against BNL cells was observed using splenocytes derived from mice treated with DC/BNL fusions (FIG. 13). Splenocytes from mice treated with both DC/BNL fusions and IL-12 showed stronger cytolytic activity against BNL cells than splenocytes from mice treated with DC/BNL fusions only. By contrast, there was no evidence of cytolytic activity against C26 colon carcinoma cells (FIG. 14).

Identification of Effector Cells Induced by Vaccination with the Fusions

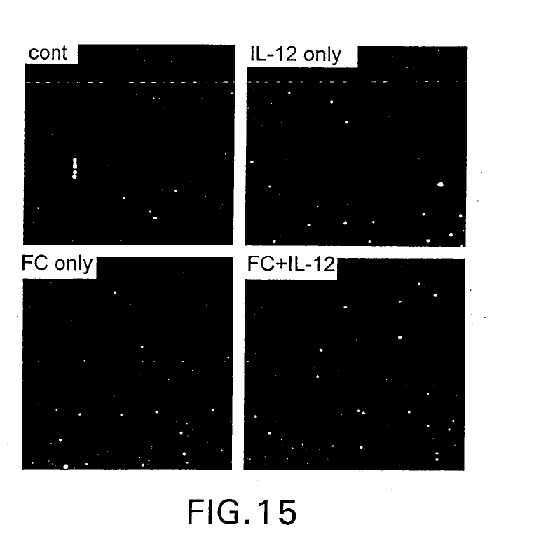

Splenocytes from mice immunized with DC/BNL fusions were examined for lytic activity against BNL cells in the presence of antibodies against CD4, CD8, H-2K^{d} and IA^{d}/I-E^{d}. Lytic activity of the splenocytes treated with antibody against CD4 was significantly reduced, while those treated with antibody against CD8 exhibited almost the same lytic activity as those treated with an isotype identical antibody, rat IgG₂ₐ (FIG. 15A). Lytic activity of the splenocytes from the fusion-treated mice was significantly inhibited when BNL cells were treated with antibody against I-A^{d}/I-E^{d}, but not H-2K^{d}. These results suggest that effector cells induced by immunization with DC/BNL fusions are CD4⁺ CTLs and the cytotoxicity is MHC class II-dependent.

Immunohistochemical Studies on BNL Tumors Growing in the Fusion-Treated Mice.

BNL tumors which grew in spite of the prior injection of DC/BNL fusions were examined by immunohistochemistry, for infiltration of CD4⁺ cells and expression of 1-Ad/I-E^{d} and for ICAM-1. In this study, DC/BNL fusions were injected subcutaneously, twice, at a two week interval. BNL cells, 10⁹/mouse, were inoculated subcutaneously 7 days after the second injection of the fusions.

When small tumors emerged, some mice were treated with 200 ng of IL-12 three times a week. The tumor was resected one day after the third administration of IL-12. CD4⁺ cells were detectable in the tumors that formed in the fusion-treated mice which had received IL-12. By contrast, few CD4⁺ cells were seen in tumors formed in mice treated with the fusions alone. I-A^{d}/I-E^{d} molecules were expressed more abundantly in BNL tumors formed in mice which had received administration of IL-12.

CD54 (Intercellular adhesion molecule 1; ICAM-1) was also expressed at higher levels on BNL tumor cells in mice treated with IL-12. These results suggest that main effector cells reactive with BNL cells induced by immunization with DC/BNL fusions were CD4⁺ CTLs. Moreover, treatment with IL-12 induces tumor cell susceptibility to CD4⁺ CTLs by enhanced expression of MHC class II and ICAM-1 molecules.

6.2.3 Discussion

DCs are potent antigen-presenting cells that can present tumor antigens to naive T cells and prime them against these antigens (Grabbe et al., 1995, Immunolo. Today, 16:117-121; Shurin, M. R., 1996, Cancer Immunol., 43:158-164). A current focus of cancer immunotherapy is the utilization of DCs as an immunotherapeutic agent. Because DCs can process and present exogenous antigens to not only CD4⁺ T cells, but also CD8+ T cells, antitumor immunity induced by loading DCs with tumor lysate or antigenic peptides carried in the context of MHC molecules on the tumor cell surface may be a promising antitumor strategy (Paglia et al., 1996, J. Exp. Med., 183:317-322; Mayordomo et al., 1995, Nat. Med., 1: 1297-1302; Celluzzi et al., 1996, J. Exp. Med., 183:283-287, Zivogel et al., 1996, J. Exp. Med., 183:87-97; Nestle et al., 1998, Nat. Med., 4:328-332; Porgador et al., 1995, J. Exp. Med., 182:255-260).

It has been reported that DCs fused with tumor cells induce antitumor immunity (Gong et al., 1997, Nat. Med. 3:558-561). In this setting, fusion cells present antigenic epitopes of tumor antigens to naive T cells and prime them against these antigens, because fusion cells simultaneously carry antigenic epitopes of the tumor cell and retain expression of MHC class I and class II molecules, co-stimulatory molecules (CD80, CD86) and intercellular adhesion molecule-1 (ICAM-1).

By fusing autologous DCs and tumor cells, obstacles to the induction of antitumor immunity such as MHC restriction, unique mutations of tumor antigens (Robbins et al., 1996, J. Exp. Med., 183:1185-1192; Brandle et al., 1996, J. Exp. Med., 183:2501-2508), and the multiplicity of tumor-specific epitopes may be overcome. Furthermore, problems of peptide-pulsed DCs, such as the low affinity of pulsed antigenic peptides to MHC molecules (Banchereau et al., 1998, Nature, 392:245-252) and the short life span of peptide-pulsed MHC class I molecules (Cella et al., 1997, Nature, 388:782-792) are not issues in fusion-based immunization. In addition, the number of BNL cells required for cell fusion is one half to one third that of DCs. A small number of requisite tumor cells is an advantage for the clinical application of fusion-based immunotherapy. Tumor cells that can be obtained at tumor biopsy might suffice as a source of fusion partners for DCs.

For the clinical application of DC/cancel cell fusions, assessment of the fusion efficacy of DCs and tumor cells by treatment with PEG and exclusion of cancer cells are important. Nonadherent cells showed DC markers, I-A^{d} and CD11c, whereas adherent cells did not, indicating that the nonadherent cell fraction contained fusion cells and DCs, and that most adherent cells were BNL cells which were not fused with DCs. In the nonadherent cell fraction, phase-contrast microscopy and scanning electron microscopy showed multi-dendritic cells larger than DCs. Two-color FACS analysis showed that approximately 30% of the PEG-treated nonadherent cells were positive for both PKH-2GL and PKH-26. Cells positive for both fluorescent dyes expressed MHC class II, CD80, CD86 and CD54 molecules which are required for antigen presentation. It is conceivable, therefore, that the fusions can present BNL tumor antigen(s) to naive T cells by means of DC capability. Immunization of BALB/c mice with DC/BNL was associated with protection against challenge with BNL cells. Moreover, splenocytes from the immunized mice showed significant lytic activity against BNL cells. By contrast, the finding that the splenocytes do not exhibit lytic activity against C26 murine colon carcinoma cells indicates that the antitumor immunity is specific for BNL cells. Mice immunized with a mixture of DCs and BNL cells, which were not treated with PEG, exhibited less protection against BNL cell challenge than did the DC/BNL fusion cells. Celluzzi, C. M. and Falo, L. J. (1998, J. Immunol, 160, 3081-5) found no difference of antitumor immunity between DC/B16 melanoma cell fusions and a mixture of DCs and B16 melanoma cells. This discrepancy might be due to differences in antigenicity between BNL HCC cells and B16 melanoma cells.

IL-12 is a heterodimeric (p35/p40) cytokine originally termed cytotoxic lymphocyte maturation factor (CLMF) (Stern et al., 1990, Proc. Natl. Acad. Sci. USA, 87:6808-6812) or natural killer cell stimulating factor (NKSF) (Kobayashi et al., 1989, J. Exp. Med., 170:827845). IL-12 plays a key role in differentiation of naive precursors to TH₁ cells to induce antitumor immunity (Tahara et al., 1995, Gene Ther., 2:96-106; Dustin et al., 1986, J. Immunol., 137:245-254; Schmitt et al., 1994, Eur. J. Immunol., 24:793-798). Dendritic cells that produce high levels of IL-12 drive naive helper T cells to differentiate to TH₁ (Macatonia et al., 1995, J. Immunol., 154:5071-5079). Splenocytes from mice treated with DC/BNL fusions in combination with IL-12 showed greater cytolytic activity against BNL cells than those treated with DC/BNL fusions alone (FIG. 14). Helper T lymphocytes stimulated by a specific antigen and co-stimulated through CD80 and CD86 express IL-12 receptor (Igarashi et al., 1998, J. Immunol., 160:1638-1646). Immunization with DCs pulsed with tumor peptide and systemic administration of IL-12 elicit effective antitumor immunity (Zitvogel et al., 1996, Anal. New York Acad. Sci., 795:284-293). IFN—Y induced by IL-12 enhances the function of proteosomes and efficacy of antigen presentation by DCs (Griffin et al., 1998, J. Exp. Med., 187:97-104) and possibly by the fusion cells. In the present studies, systemic administration of IL-12 alone had no effect against pre-established BNL tumors. Nonspecific activation of CTLs or NK cells by treatment with IL-12 is apparently not sufficient to induce tumoricidal activity. The present studies also demonstrate that induction of specific CTLs by immunization with DC/tumor cell fusions and activation of the induced CTLs by IL-12 produce effective and tumor-specific antitumor immunity. It is also conceivable that DC-tumor cell fusions can not produce sufficient IL-12 to induce Th1 condition. IL-12 produced and released from DCs presenting a specific antigen to naive T helper cells activates Th1 cells (Macatonia et al., 1995, J. Immunol., 154:5071-5079). If the ability of DC to produce IL-12 is attenuated by cell fusion, systemic administration of IL-12 to the fusion-immunized host may contribute to the development of Th1 cells and generation of specific CTLs. Another possibility is that antigen presentation by the fusions induces a Th2 response and secretion of IL-10, an inhibitor of IL-12 production (Hino et al., 1996, Eur. J. Immunol., 26:623-628). Systemic administration of IL-12 could also inhibit Th2 response and generate tumoricidal CTLs.

Cytolytic activity of splenocytes from mice treated with the fusions was inhibited by treatment of the splenocytes with antibody against CD4 and treatment of the target cells with antibody against I-A^{d}/I-E^{d}. These findings suggest that BNL-specific effector cells are CD4⁺ CTLs and cytotoxicity is dependant on MHC class II (Shinohara N., 1987, Cellular Immunol., 107:395-407; Ozdemirli et al., 1992, J. Immunol., 149:1889-1885; Yasukawa et al., 1993, Blood, 81:1527-1534). DCs present specific tumor antigen to CD8⁺ CTLs and tumoricidal activity is MHC class I dependent (Porgador et al., 1995, J. Exp. Med., 182:255-260). Although CD4⁺ CTLs are uncommon, CD4⁺ CTLs work in almost the same manner as CD8⁺ CTLs (Yasukawa et al., 1993, Blood, 81:1527-1534). In this study, cytolytic activity was not inhibited by treatment of effector cells with antibodies against CD8 nor treatment of the target cells with antibody against MHC class I. Expression of MHC class II (1-A^{d}/I-E^{d}) molecules on BNL tumor in vivo was greatly enhanced when BNL bearing mice were treated with IL-12. This response may be due to the induction of interferon-γ, tumor necrosis factor (TNF) or interleukin-1 (Gately et al., 1994, Int. Immunol., 6:157-167; Nastala et al., 1994, J. Immunol., 153:1697-1706). Enhanced expression of MHC class II molecules increases exposure of antigenic peptides from BNL tumor antigens to CD4⁺ CTLs. Furthermore, expression of ICAM-1 by BNL tumor tissue was more enhanced by treatment of the tumor-bearing mice with IL-12. This effect could also be due to the effect of IFN-γ or IL-1 directly or indirectly induced by IL-12 (Dustin et al., 1986, J. Immunol., 137:245-254). These results suggest that CTLs are able to attach to endothelial cells of the tumor and migrate into the tumor tissue more efficiently by IL-12 treatment, leading to enhanced antitumor activity against established lesions.

The development and frequent recurrence of multicentric HCC are serious problems in patients with virus-induced cirrhosis. Therefore, methods of preventing the development of HCC are needed. Small HCCs can be detected with ultrasonography and curatively treated with percutaneous ethanol injection therapy or surgical resection. To prevent the development of new HCCs and treat remaining micrometastases, tumor cells obtained at biopsy or resection can be fused with DCs. Thus, as demonstrated in this example, immunization with fusions of autologous DCs and tumor cells combined with IL-12 administration is a promising method for the treatment of HCC.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

What is claimed is:

1. A method of treating or preventing a cancer in a patient, said method comprising administering to the patient a therapeutically effective amount of
(a) fusion cells formed by fusing (i) autologous cancer cells and (ii) dendritic cells that have the same class I MHC haplotype as said patient; and
(b) interleukin-12.

2. The method of claim 1, wherein said cancer cells are obtained from said patient by surgery or biopsy.

3. The method of claim 1, wherein said cancer cells are obtained from said patient at most 30 minutes before the fusion of said cancer cell and said dendritic cell.

4. The method of claim 1, wherein said cancer cells are obtained from said patient at most 60 minutes before the fusion of said cancer cell and said dendritic cell.

5. The method of claim 1, wherein said cancer cells are obtained from said patient at most 120 minutes before the fusion of said cancer cell and said dendritic cell.

6. The method of claim 1, wherein said cancer cells are obtained from said patient at most 5 hours before the fusion of said cancer cell and said dendritic cell.

7. The method of claim 1, wherein said cancer cells are cultivated in cell culture before fusion to said dendritic cell.

8. The method of claim 1, wherein said fusion cells are obtained by incubating said dendritic cell and said cancer cells in a medium comprising from about 0.5% to about 25% polyethyleneglycol.

9. The method of claim 8, wherein the medium comprises about 2.5% polyethyleneglycol

10. The method of claim 8, wherein said dendritic cell and said cancer cell are incubated overnight.

11. The method of claim 1, wherein said fusion cells are washed before administration to the patient.

12. The method of claim 1, wherein said fusion cells are formed by incubating cancer cells and dendritic cells together at a ratio of about 1 cancer cell per 10 dendritic cells.

13. The method of claim 12, wherein said fusion cells are formed by incubating cancer cells and dendritic cells together at a ratio of 3 cancer cells per dendritic cell.

14. The method of claim 1, wherein said cancer cells are irradiated at about 50 to 1,000 Gy.

15. The method of claim 14, wherein said cancer cells are irradiated at about 300 Gy.

16. The method of claim 1, wherein the amount of interleukin-12 administered is between 10 ng and 100 ng interleukin-12 per kg body weight of the patient.

17. The method of claim 1, wherein the amount of interleukin-12 administered is about 30 ng interleukin-12 per kg of body weight of patient.

18. The method of claim 17, wherein said interleukin-12 is administered 6 to 12 times to the patient.

19. The method of claim 1, wherein said dendritic cells are obtained from blood monocytes from the patient.

20. The method of claim 19, wherein the method for obtaining blood monocytes comprises culturing leukocytes obtained from the patient in a medium comprising from about 1% to about 10% serum of the patient.

21. The method of claim 19, wherein the method for obtaining blood monocytes comprises culturing leukocytes obtained from the patient in a medium comprising GM-CSF and IL-4.

22. The method of claim 21, wherein the leukocytes are leukocytes with high adherent capacity.

23. The method of claim 21, wherein the concentration of GM-CSF is between about 10 and 100 ng/ml and the concentration of IL-4 is between about 10 and 100 U/ml.

24. The method of claim 23, wherein the concentration of GM-CSF is about 10 ng/ml and the concentration of IL-4 is about 30 U/ml.

25. The method of claim 21, wherein the medium further comprises TNF-α.

26. The method of claim 25, wherein the concentration of TNF-α is about 20 ng/ml.

27. The method of claim 25, wherein the TNF-α is added after 5 days of culturing.

28. The method of claim 21, wherein the leukocytes are cultured for about 7 to 10 days.

29. The method of claim 28, wherein the leukocytes are cultured for 7 days.

30. The method of claim 1, wherein said interleukin-12 is recombinant human interleukin-12.

31. The method of claim 1, wherein, prior to administration, the effect of administering interleukin-12 alone is tested by a prick test.

32. The method of claim 1, wherein said method further comprises one or more tests on the patient, wherein the test is selected from the group consisting of hematological test, urinanalysis, fecal test, pregnancy test, and imaging examination.

33. The method of claim 1, wherein said fusion cells are cultured in a medium comprising GM-CSF, IL-4, and TNF-α.

34. The method of claim 33, wherein the concentration of GM-CSF is about 10 ng/ml to about 100 ng/ml, the concentration of IL-4 is about 10 U/ml to about 100 U/ml, and the concentration of TNF-α is about 10 ng/ml to about 100 ng/ml.

35. The method of claim 34, wherein the concentration of GM-CSF is 10 ng/ml, the concentration of IL-4 is 30 U/ml, and the concentration of TNF-α is 20 U/ml.

36. The method of claim 1, wherein said fusion cells are free of microbial contamination.

37. The method of claim 1, wherein said method further comprises administering an antipyretic.

38. The method of claim 37, wherein the antipyretic is salicylic acid, indomethacin, sodium indomethacin trihydrate, salicylamide, naproxen, colchicine, fenoprofen, sulindac, diflunisal, diclofenac, indoprofen or sodium salicylamide.

39. The method of claim 1, wherein said method further comprises administering an immunosuppressant.

40. The method of claim 39, wherein the immunosuppressant is selected from the group consisting of a glucocorticoid, an hydroorotate dehydrogenase inhibitor, a myelin basic protein, an anti-Fc receptor monoclonal antibody, an anti-IL2 monoclonal antibody, a 5-lipoxygenase inhibitor, a phosphatidic acid synthesis antagonist, a platelet activating factor antagonist, a selectin antagonist, an interleukin-10 agonist, a peptigen agent, a protein kinase C inhibitor, a phosphodiesterase IV inhibitor, a single chain antigen binding protein, a complement factor inhibitor, a spirocyclic lactam, a 5-hydroxytryptamine antagonist, and an anti-TCR monoclonal antibody.

41. The method of claim 39, wherein the immunosuppressant is methylprednisolone, 7-capaxone, CHI-621, dacliximab, buspirone, castanospermine, CD-59, CMI-392, ebselen, edelfosine, enlimomab, galaptin, ICAM4, macrocylic lactone, methoxatone, mizoribine, OX-19, PG-27, sialophorin, sirolimus, CD5 gelonin or TOK-8801.

42. The method of claim 1, wherein the fusion cells are subcutaneously injected into the groin area of the patient in a suspension comprising physiological saline.

43. The method of claim 1, wherein said therapeutically effective amount of fusion cells is from about 1×10⁵ to about 5×10⁵ fusion cells.

44. The method of claim 43, wherein about 1×10⁵ to about 5×10⁵ fusion cells are administered 3 to 6 times.

45. The method of claim 1, wherein said fusion cells and said interleukin-12 are administered in up to 6 cycles.

46. The method of claim 45, wherein said cycle consists of a first week and a second week.

47. The method of clam 46, wherein (i) said first week is characterized by administering separately said therapeutically effective amount of fusion cells, followed by administering a first said therapeutically effective amount of interleukin-12, and followed by administering a second said therapeutically effective amount of interleukin-12, and wherein (ii) said second week is characterized by withdrawal of said fusion cells and said interleukin-12.

48. The method of claim 1, wherein the cancer is selected from the group consisting of renal cell carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemias, acute lymphocytic leukemia, acute myelocytic leukemia; chronic leukemia, polycythemia vera, lymphoma, multiple myeloma, Waldenstrom's macroglobulinemia, and heavy chain disease.
